(19) United States
(12) Patent Application Publication
(10) Pub. No.: US 2023/0279422 A1
(43) Pub. Date: Sep. 7, 2023

(54) RECOMBINANT AAV VECTORS FOR TREATING NERVOUS SYSTEM DISEASES

(71) Applicants: ICM (INSTITUT DU CERVEAU ET DE LA MOELLE ÉPINIÈRE), Paris (FR); INSERM (INSTITUT NATIONAL DE LA SANTÉ ET DE LA RECHERCH MÉDICALE), Paris Cedex 13 (FR); CENTRE NATIONAL DE LA RECHERCHE SCIENTIFIQUE, Paris (FR); APHP (ASSISTANCE PUBLIQUE - HÔPITAUX DE PARIS), Paris (FR); SORBONNE UNIVERSITE, Paris (FR)

(72) Inventors: Françoise PIGUET, Paris (FR); Caroline SEVIN, Paris (FR)

(73) Assignees: ICM (INSTITUT DU CERVEAU ET DE LA MOELLE ÉPINIÈRE), Paris (FR); INSERM (INSTITUT NATIONAL DE LA SANTÉ ET DE LA RECHERCH MÉDICALE), Paris Cedex 13 (FR); CENTRE NATIONAL DE LA RECHERCHE SCIENTIFIQUE, Paris (FR); APHP (ASSISTANCE PUBLIQUE - HÔPITAUX DE PARIS), Paris (FR); SORBONNE UNIVERSITE, Paris (FR)

(21) Appl. No.: 17/749,501

(22) Filed: May 20, 2022

Related U.S. Application Data
(60) Provisional application No. 63/316,598, filed on Mar. 4, 2022.

(51) Int. Cl. C12N 15/86; A61K 38/46; A61P 25/00

(52) U.S. Cl. CPC C12N 15/86; A61K 38/465; C12Y 301/06008; A61P 25/00; C12N 2750/14122; C12N 2750/14143; A61K 48/00

(57) ABSTRACT

The use of recombinant AAV vectors expressing a molecule of interest in a method for treating diseases or conditions affecting the nervous system in a subject in need thereof, or in a method for increasing or inducing expression of the molecule of interest in the nervous system of a subject.

### FIELD

The present invention relates to the use of recombinant AAV vectors expressing a molecule of interest in a method for treating diseases or conditions affecting the nervous system in a subject in need thereof, or in a method for increasing or inducing expression of the molecule of interest in the nervous system of a subject.

### BACKGROUND

Recombinant adeno-associated viruses (rAAVs) are increasingly used as delivery vehicles because they display several advantages: low pathogenicity, stability, ability to transduce both dividing and non-dividing cells, as well as non-integrating expression in vivo. However, most of AAV serotypes fail to cross the blood-brain barrier, and thereof cannot be administered non-invasively to treat diseases from the nervous system. Hence, AAV vectors, especially AAV9, have been injected through intracerebral or intrathecal administration routes to efficiently transduce the nervous system.

To circumvent this problem, capsid variants derived from AAV capsids, especially AAV9 capsids, have been developed to try to increase the crossing of the blood-brain barrier and the transduction efficiency in the nervous system. As an example, in mouse model, the AAV9 variant vectors AAV-PHP.B and AAV-PHP.eB have been shown to efficiently transduce across the peripheral and central nervous systems when said vectors were administered intravenously (Chan et al., Engineered AAVs for efficient noninvasive gene delivery to the central and peripheral nervous systems, Nat Neurosci. 2017 August; 20(8):1172-1179, and Deverman et al., Cre-dependent selection yields AAV variants for widespread gene transfer to the adult brain, Nat Biotechnol. 2016 February; 34(2):204-9).

AAV-PHP.B and AAV-PHP.eB have been then applied across a wide range of neuroscience experiments in mice, including genetic deficit correction and neurological disease modeling. However, at odds with rodent data, intravenously delivered AAV-PHP.B and AAV-PHP.eB did not demonstrate the same capabilities of blood-brain barrier crossing and transduction efficiency in non-human primates (NHPs) (Matsuzaki et al., Intravenous Administration of the Adeno-Associated virus-PHP.B Capsid Fails to Upregulate Transduction Efficiency in the Marmoset Brain, Neurosci Lett. 2018 Feb. 5; 665:182-188; Hordeaux et al., 2018, The Neurotropic Properties of AAV-PHP.B Are Limited to C57BL/6J Mice, Mol Ther. 2018 Mar. 7; 26(3):664-668 and Goersten et al., AAV capsid variants with brain-wide transgene expression and decreased liver targeting after intravenous delivery in mouse and marmoset, Nat Neurosci. 2022 January; 25(1):106-115). One of the hypotheses put forward to explain these discrepancies between species is that the AAV-PHP.B family requires the presence of the receptor LY6A, which is absent in primates (Huang et al., Delivering genes across the blood-brain barrier: LY6A, a novel cellular receptor for AAV-PHP.B capsids, PLoS One. 2019 Nov. 14; 14(11):e0225206). Thereof, the species-specific tropism of the AAV-PHP.B capsids has greatly reduced their appeal for human nervous system gene therapy and new variants capable of efficiently transducing the nervous system of NHPs have been developed (Goersten et al., see above).

Here, the Inventors have discovered that the intravenous injection of AAV-PHP.eB vectors comprising a nucleic acid sequence encoding arylsulfatase A (ARSA) is able to improve metachromatic leukodystrophy (MLD) pathophysiology in symptomatic mice. Surprisingly, the Inventors have also demonstrated that the intravenous injection of said AAV-PHP.eB vectors in NHP induced expression of ARSA in the nervous system of the animals with no signs of toxicity. These data provide strong evidence that, unlike what was thought in the art, AAV-PHP.B family vectors, such as AAV-PHP.eB vectors, could be used in human therapy with non-invasive administration to treat nervous system diseases, such as MLD.

### SUMMARY

The present invention relates to a method for treating a disease or condition affecting the nervous system in a subject in need thereof, comprising administrating to said subject a recombinant AAV vector comprising a nucleic acid sequence encoding a molecule of interest,
wherein said recombinant AAV vector comprises an AAV capsid protein comprising the amino acid sequence TLAVPFK (SEQ ID NO: 1),
wherein said recombinant AAV vector is administered buccally, nasally, orally, rectally, intramuscularly, intravenously or subcutaneously or a combination thereof to said subject, and
wherein said disease or condition, and said molecule of interest are one of the followings combinations:
   i) the disease or condition is metachromatic leukodystrophy (MLD), and the molecule of interest is arylsulfatase A (ARSA);
   ii) the disease or condition is Alzheimer's disease, Huntington's disease, Parkinson's disease, spinocerebellar ataxia or glioblastoma, and the molecule of interest is cholesterol 24-hydroxylase,
   iii) the disease or condition is mucopolysaccharidosis type III, and the molecule of interest is N-acetyl-alpha-glucosaminidase.

In one embodiment, said subject is a primate. In one embodiment, said subject is a human

In one embodiment, said AAV vector is a variant AAV9 vector.

In one embodiment, the amino acid sequence TLAVPFK (SEQ ID NO: 1) is inserted between the amino acid residues 588-589 of the AAV9 capsid protein of sequence SEQ ID NO: 2.

In one embodiment, said AAV9 capsid protein further comprises at least one of the mutations A587D and Q588G. In one embodiment, said AAV9 capsid protein further comprises the two mutations A587D and Q588G.

In one embodiment, the disease or condition is metachromatic leukodystrophy (MLD) and the molecule of interest is ARSA. In one embodiment, the MLD is selected from the group consisting of the late infantile form, the juvenile form, and the adult form.

In one embodiment, said subject is symptomatic. In one embodiment, said subject presents at least one of the following symptoms: sulfatide storage, myelin abnormalities on MRI, neuroinflammation, motor impairment and/or coordination loss.

In one embodiment, said method further comprises a step of exposing the subject to ultrasounds.

The present invention further relates to a method for increasing or inducing expression of a molecule of interest in the nervous system of a subject, comprising administrating to said subject a recombinant AAV vector comprising a nucleic acid sequence encoding the molecule of interest,
wherein said recombinant AAV vector comprises an AAV capsid protein comprising the amino acid sequence TLAVPFK (SEQ ID NO: 1),
wherein said recombinant AAV vector is administered buccally, nasally, orally, rectally, intramuscularly, intravenously or subcutaneously to said subject, and
wherein said molecule of interest is ARSA.

In one embodiment, said subject is a primate. In one embodiment, said subject is a human.

In one embodiment, said AAV vector is a variant AAV9 vector.

In one embodiment, the amino acid sequence TLAVPFK (SEQ ID NO: 1) is inserted between the amino acid residues 588-589 of the AAV9 capsid protein of sequence SEQ ID NO: 2.

In one embodiment, said AAV9 capsid protein further comprises the two mutations A587D and Q588G.

In one embodiment, said method further comprises a step of exposing the subject to ultrasounds.

### BRIEF DESCRIPTION OF THE DRAWINGS

FIG. 1(A-B) is a combination of two histograms showing that AAVPHP.eB-hARSA-HA efficiently transduce central nervous system. A: Biodistribution of the AAVPHP.eB-hARSA-HA in central nervous system. B: Biodistribution of the AAVPHP.eB-hARSA-HA in peripheral organs.

FIG. 2(A-B) is a combination of two histograms showing ARSA activity and expression in several brain regions. A: ARSA activity in several brain regions and spinal cord in 9-month-old wild-type (n=3), untreated (n=7) and treated KO ARSA (n=5) mice with AAVPHP.eB-hARSA-HA. B: Arylsulfatase A (ARSA) expression (ng/mg protein) assessed by ELISA in several brain regions and spinal cord in 9-month-old wild-type (n=3), untreated (n=3) and treated KO ARSA (n=5) mice with AAVPHP.eB-hARSA-HA. Data are represented as mean±SEM.

FIG. 3(A-D) is combination of four histograms showing the correction of sulfatide storage in brain and spinal cord of treated KO-ARSA mice, 3 months after treatment. A-D: Quantification of sulfatide storage per mm2 in cortex (A), corpus callosum (B), fimbria (C) and spinal cord (D) of WT (n=3), untreated (NT, n=6-8) and treated (AAV, n=5) KO ARSA mice. Data are represented as mean±SEM. ***p<0.001; ****p<0.0001.

FIG. 4 (A-D) is a combination of four histograms showing the correction of astrogliosis in brain and spinal cord of treated KO-ARSA mice, 3 months after treatment. A-D: Quantification of GFAP-positive cells per mm2 in cortex (A), corpus callosum (B), cerebellum (C) and spinal cord (D) of WT (n=3), untreated (NT, n=6-8) and treated (AAV, n=5) KO ARSA mice. Data are represented as mean±SEM. *p<0.05; **p<0.01.

FIG. 5(A-B) is a combination of two histograms showing the correction of microgliosis in cortex and corpus callosum of treated KO-ARSA mice, 3 months after treatment. A-B: Quantification of Iba1-positive cells per mm2 in cortex (A) and corpus callosum (B) of WT (n=3), untreated (NT, n=6-8) and treated (AAV, n=5) KO ARSA mice. Data are represented as mean±SEM.*p<0.05; **p<0.01.

FIG. 6 is a histogram showing Arylsulfatase A (ARSA) expression (ng/mg protein) assessed by ELISA in the cerebrospinal fluid of a monkey having received an intravenous injection of AAV-PHP.eB-ARSA, as compared to a control monkey, at baseline, three weeks after injection and six weeks after injection.

FIG. 7 is a histogram showing Arylsulfatase A (ARSA) expression (ng/mg protein) assessed by ELISA in tissues from the CNS and peripheral organs of a monkey having received an intravenous injection of AAV-PHP.eB-ARSA, as compared to a control monkey, at 6 weeks after injection.

FIG. 8 is a histogram showing the quantification of the sulfatides storage by Alcian blue staining in WT and KO ARSA mice at 6 and 9 months in nervous system and peripheral tissues to evaluate the sulfatide storage at injection time.

FIG. 9 is a histogram showing the quantification of the microgliosis by Iba1 staining in WT and KO ARSA mice at 6 and 9 months in nervous system tissues to evaluate the neuroinflammation at injection time.

FIG. 10 is a histogram showing the quantification of sulfatide isoforms (C16, C16OH, C18, C18OH, C20, C20OH, C22, C23, C22OH, C24:1, C24, C23OH, C24:1OH, C24OH, C26, C26OH) in WT and KO ARSA mice and KO ARSA mice treated with 5·10¹¹ vg total of AAVPHP.eB-hARSA-HA at 6 or 9 months in the cerebellum.

FIG. 11 is a histogram showing the quantification of ARSA activity in nervous system tissues in non-human primates (NHP) treated with an intravenous administration of 1·10¹³ vg total of AAVPHP.eB-hARSA-HA or an intrathecal administration of 1·10¹³ vg total of AAVPHP.eB-hARSA-HA.

FIG. 12 is a histogram showing the quantification of ARSA activity in peripheral tissues in non-human primates (NHP) treated with an intravenous administration of 1·10¹³ vg total of AAVPHP.eB-hARSA-HA or an intrathecal administration of 1·10¹³ vg total of AAVPHP.eB-hARSA-HA. The following structures: cervical and inguinal lymphnodes, ovary, quadri, VB, heart and lung tissues were not collected for Monkey 1 (IV).

### DETAILED DESCRIPTION

In the present invention, the following terms have the following meanings:

“Adeno-associated virus” or “AAV”: refers to a member of the parvovirus family of single-stranded small DNA viruses that require a helper virus, such as adenovirus or herpes simplex virus, for replication. AAV contains two genes, rep and cap, that are required for its replication.

“About”: preceding a figure encompasses plus or minus 10%, or less, of the value of said figure. It is to be understood that the value to which the term “about” refers is itself also specifically, and preferably, disclosed.

“ARSA” or “Arylsulfatase A” refers to the enzyme that catalyzes the first step in the degradation pathway of 3-O-sulfogalactosylceramides (sulfatides). An example of human ARSA is provided with the reference UniProtKB—P15289.

“Blood-brain barrier”: refers to the selective permeable membrane that regulates the passage of molecules into the extracellular fluid of the central nervous system.

“Cholesterol 24-hydroxylase” (also known as “cholesterol 24S-hydroxylase”, or “cholesterol 24-monooxygenase”): refers to an enzyme that catalyzes the conversion of cholesterol to 24S-hydroxycholesterol. This enzyme is a member of the cytochrome P450 (CYP) superfamily of enzymes. Said enzyme is encoded by the CYP46A1 gene. An example of human cholesterol 24-hydroxylase is provided with the reference UniProtKB—Q9Y6A2.

“Identity” or “identical”: when used in a relationship between the sequences of two or more amino acid sequences, or of two or more nucleic acid sequences, refers to the degree of sequence relatedness between amino acid sequences or nucleic acid sequences, as determined by the number of matches between strings of two or more amino acid residues or nucleic acid residues. Identity of related amino acid sequences or nucleic acid sequences can be readily calculated by known methods. Such methods include, but are not limited to, those described in Lesk A. M. (1988). Computational molecular biology: Sources and methods for sequence analysis. New York, N.Y.: Oxford University Press; Smith D. W. (1993). Biocomputing: Informatics and genome projects. San Diego, Calif.: Academic Press; Griffin A. M. & Griffin H. G. (1994). Computer analysis of sequence data, Part 1. Totowa, N.J.: Humana Press; von Heijne G. (1987). Sequence analysis in molecular biology: treasure trove or trivial pursuit. San Diego, Calif.: Academic press; Gribskov M. R. & Devereux J. (1991). Sequence analysis primer. New York, N.Y.: Stockton Press; Carillo et al., 1988. SIAM J Appl Math. 48(5):1073-82. Preferred methods for determining identity are designed to give the largest match between the sequences tested. Methods of determining identity are described in publicly available computer programs. Preferred computer program methods for determining identity between two sequences include the GCG program package, including GAP (Genetics Computer Group, University of Wisconsin, Madison, Wis.; Devereux et al., 1984. Nucleic Acids Res. 12(1 Pt 1):387-95), BLASTP, BLASTN, and FASTA (Altschul et al., 1990. J Mol Biol. 215(3):403-10). The BLASTX program is publicly available from the National Center for Biotechnology Information (NCBI) and other sources (BLAST Manual, Altschul et al. NCB/NLM/NIH Bethesda, Md. 20894). The well-known Smith Waterman algorithm may also be used to determine identity.

“Lysosomal storage diseases” or “LSD”: refers to inherited metabolic disorders characterized by defects in lysosomal function and resulting in intracellular accumulation of unmetabolized substrates.

“Mammal”: refers to any mammal, including humans, domestic and farm animals, and zoo, sports, or pet animals, such as dogs, cats, cattle, horses, sheep, pigs, goats, rabbits, etc.

“NAGLU” or “N-acetyl-alpha-glucosaminidase” refers to the enzyme that degrades heparan sulfate by hydrolysis of terminal N-acetyl-D-glucosamine residues in N-acetyl-alpha-D-glucosaminides. An example of a human NAGLU is provided with the reference UniProtKB—P54802.

“Nervous system”: refers to the organized network of nerve tissue in the body. In vertebrates, it includes the central nervous system (CNS) (i.e. the brain and spinal cord) and the peripheral nervous system (i.e. dorsal roots ganglia and nerves that extend from the spinal cord to the rest of the body).

“Primate”: refers to any member of the biological order Primates, the group that contains all the species commonly related to the lemurs, monkeys, and apes, with the latter category including humans.

“Serotypes”: refers to groups within a single species of microorganisms, such as bacteria or viruses, which share distinctive surface structures. To date, regarding AAV, 12 serotypes have been identified and include AAV1, AAV2, AAV3, AAV4, AAV5, AAV6, AA7, AAV8, AAV9, AAVrh.10, AAV11 and AAV12 serotypes.

“Subject”, as used herein, refers to a mammal, preferably a human. In one embodiment, a subject may be a “patient”, i.e., a warm-blooded animal, more preferably a human, who/which is awaiting the receipt of, or is receiving medical care or was/is/will be the object of a medical procedure, or is monitored for the development of a disease.

“Therapeutically effective amount”: refers to the level or amount of an AAV vector as described herein that is aimed at, without causing significant negative or adverse side effects to the target, (1) delaying or preventing the onset of a disease, disorder, or condition; (2) slowing down or stopping the progression, aggravation, or deterioration of one or more symptoms of the disease, disorder, or condition; (3) bringing about ameliorations of the symptoms of the disease, disorder, or condition; (4) reducing the severity or incidence of the disease, disorder, or condition; or (5) curing the disease, disorder, or condition. A therapeutically effective amount may be administered prior to the onset of the disease, disorder, or condition, for a prophylactic or preventive action. Alternatively or additionally, the therapeutically effective amount may be administered after initiation of the disease, disorder, or condition, for a therapeutic action.

“Treating” or “treatment”: refers to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. A subject is successfully “treated” for a disease or condition if, after receiving a therapeutic amount of an AAV vector according to the methods of the present invention, the subject shows observable and/or measurable reduction in or absence of one or more of the following: reduction in the number of pathogenic cells; and/or relief to some extent of one or more of the symptoms associated with the specific disease or condition; reduced morbidity and mortality, and improvement in quality of life issues. The above parameters for assessing successful treatment and improvement in the disease are readily measurable by routine procedures familiar to a physician.

The present invention relates to a recombinant AAV vector comprising a nucleic acid sequence encoding a molecule of interest.

As used herein, recombinant AAV vectors are AAV that are modified to comprise a nucleic acid sequence encoding a molecule of interest.

In one embodiment, the AAV vector according to the present invention has a serotype selected from the group consisting of AAV1, AAV2, AAV3, AAV4, AAV5, AAV6, AAV7, AAV8, AAV9, AAVrh.10, AAV11, AAV12, and variants thereof. In one embodiment, the AAV vector according to the present invention has a variant AAV9 serotype.

As used herein, an AAV variant refers to a non-natural AAV derived from AAV1, AAV2, AAV3, AAV4, AAV5, AAV6, AAV7, AAV8, AAV9, AAVrh.10, AAV11 or AAV12.

In one embodiment, the AAV vector according to the present invention comprises a modified AAV capsid. It is known in the art that AAV capsids comprise AAV capsid proteins, such as VP1, and VP2 and VP3. Thus, in one embodiment, said AAV vector comprises modified AAV capsid proteins (e.g. VP1, VP2 and/or VP3) to increase the crossing of the blood-brain barrier.

In one embodiment, the AAV vector according to the present invention comprises a modified AAV capsid protein, such as a modified AAV1, AAV2, AAV3, AAV4, AAV5, AAV6, AAV7, AAV8, AAV9, AAVrh.10, AAV11 or AAV12 capsid protein, preferably a modified AAV9 capsid protein.

Examples of modifications of the AAV capsid protein include, without limitation, insertion, substitution and/or deletion of amino acids.

In one embodiment, the AAV capsid protein comprises a heterologous amino acid sequence that increases the crossing of the blood-brain barrier. In one embodiment, the AAV capsid protein, preferably an AAV9 capsid protein, comprises the amino acid sequence TLAVPFK (SEQ ID NO: 1).

In one embodiment, the amino acid sequence TLAVPFK (SEQ ID NO: 1) is inserted between the amino acid residues 588-589 of the AAV9 capsid protein of SEQ ID NO: 2.

SEQ ID NO: 2
MAADGYLPDWLEDNLSEGIREWWALKPGAPQPKANQQHQDNARGLVLP
GYKYLGPGNGLDKGEPVNAADAAALEHDKAYDQQLKAGDNPYLKYNHA
DAEFQERLKEDTSFGGNLGRAVFQAKKRLLEPLGLVEEAAKTAPGKKR
PVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVI
TTSTRTWALPTYNNHLYKQISNSTSGGSSNDNAYFGYSTPWGYFDFNR
FHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSL
DRLMNPLIDQYLYYLSKTINGSGQNQQTLKFSVAGPSNMAVQGRNYIP
GPSYRQQRVSTTVTQNNNSEFAWPGASSWALNGRNSLMNPGPAMASHK
EGEDRFFPLSGSLIFGKQGTGRDNVDADKVMITNEEEIKTTNPVATES
YGQVATNHQSAQAQAQTGWVQNQGILPGMVWQDRDVYLQGPIWAKIPH
TDGNFHPSPLMGGFGMKHPPPQILIKNTPVPADPPTAFNKDKLNSFIT
QYSTGQVSVEIEWELQKENSKRWNPEIQYTSNYYKSNNVEFAVNTEGV
YSEPRPIGTRYLTRNL

In one embodiment, the AAV vector according to the present invention comprises a variant AAV9 capsid protein of SEQ ID NO: 3. An example of such AAV vector is AAV-PHP.B.

SEQ ID NO: 3
MAADGYLPDWLEDNLSEGIREWWALKPGAPQPKANQQHQDNARGLVLP
GYKYLGPGNGLDKGEPVNAADAAALEHDKAYDQQLKAGDNPYLKYNHA
DAEFQERLKEDTSFGGNLGRAVFQAKKRLLEPLGLVEEAAKTAPGKKR
PVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVI
TTSTRTWALPTYNNHLYKQISNSTSGGSSNDNAYFGYSTPWGYFDFNR
FHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSL
DRLMNPLIDQYLYYLSRTINGSGQNQQTLKFSVAGPSNMAVQGRNYIP
GPSYRQQRVSTTVTQNNNSEFAWPGASSWALNGRNSLMNPGPAMASHK
EGEDRFFPLSGSLIFGKQGTGRDNVDADKVMITNEEEIKTTNPVATES
YGQVATNHQSAQTLAVPFKAQAQTGWVQNQGILPGMVWQDRDVYLQGP
IWAKIPHTDGNFHPSPLMGGFGMKHPPPQILIKNTPVPADPPTAFNKD
KLNSFITQYSTGQVSVEIEWELQKENSKRWNPEIQYTSNYYKSNNVEF
AVNTEGVYSEPRPIGTRYLTRNL

An example of a nucleic acid sequence encoding the variant AAV9 capsid protein as described hereinabove is SEQ ID NO: 13.

In one embodiment, the AAV capsid protein further comprises mutations, such as one or more substitution(s) of amino acids. In one embodiment, the AAV capsid protein further comprises substitutions of the amino acid residues 587 and 588 in SEQ ID NO: 2.

In one embodiment, the AAV capsid protein further comprises at least one of the two mutations A587D (i.e. the replacement of the Alanine in position 587 to an Aspartate) and Q588G (i.e. the replacement of the Glutamine in position 588 to a Glycine) in SEQ ID NO: 2. In one embodiment, the AAV capsid protein further comprises the two mutations A587D and Q588G in SEQ ID NO: 2.

In one embodiment, the AAV vector according to the present invention comprises a variant AAV9 capsid protein of SEQ ID NO: 4. An example of such AAV vector is AAV-PHP.eB.

SEQ ID NO: 4
MAADGYLPDWLEDNLSEGIREWWALKPGAPQPKANQQHQDNARGLVLP
GYKYLGPGNGLDKGEPVNAADAAALEHDKAYDQQLKAGDNPYLKYNHA
DAEFQERLKEDTSFGGNLGRAVFQAKKRLLEPLGLVEEAAKTAPGKKR
PVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVI
TTSTRTWALPTYNNHLYKQISNSTSGGSSNDNAYFGYSTPWGYFDFNR
FHCHFSPRDWQRLINNNWGFRPKRLNFKLFNIQVKEVTDNNGVKTIAN
NLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSL
DRLMNPLIDQYLYYLSRTINGSGQNQQTLKFSVAGPSNMAVQGRNYIP
GPSYRQQRVSTTVTQNNNSEFAWPGASSWALNGRNSLMNPGPAMASHK
EGEDRFFPLSGSLIFGKQGTGRDNVDADKVMITNEEEIKTTNPVATES
YGQVATNHQSDGTLAVPFKAQAQTGWVQNQGILPGMVWQDRDVYLQGP
IWAKIPHTDGNFHPSPLMGGFGMKHPPPQILIKNTPVPADPPTAFNKD
KLNSFITQYSTGQVSVEIEWELQKENSKRWNPEIQYTSNYYKSNNVEF
AVNTEGVYSEPRPIGTRYLTRNL

An example of a nucleic acid sequence encoding the variant AAV9 capsid protein as described hereinabove is SEQ ID NO: 14.

In one embodiment, the AAV vector according to the present invention further comprises a promoter. Said promoter is capable of initiating the transcription of the nucleic acid sequence encoding a molecule of interest in a target cell, preferably a human cell, preferably a human cell from the nervous system.

Examples of target cells from the nervous system, include, without limitation, glia such as astrocytes or microglia, neurons and oligodendrocytes.

In one embodiment, said promoter is ubiquitous, meaning that the promoter is expressed in a wide range of cell-types. Said promoter may be used for inducing the expression of the molecule of interest in a wide range of cells.

Examples of promoters that may be used in the present invention include, but are not limited to, a phosphoglycerate kinase (PGK) promoter, a SV40 early promoter, a mouse mammary tumor virus LTR promoter, an adenovirus major late promoter (Ad MLP), a herpes simplex virus (HSV) promoter, a cytomegalovirus (CMV) promoter such as the CMV immediate early promoter region (CMVIE) or the CMV early enhancer/chicken β-actin (CAG) promoter, a rous sarcoma virus (RSV) promoter, ARSA promoter, truncated CBA hybrid (CBh) promoter, EF1 (elongation factor 1) promoter, synthetic promoters, hybrid promoters.

In one embodiment, said promoter is a CAG promoter. In one embodiment, said promoter is ARSA promoter.

In one embodiment, said promoter is specific for the nervous system, meaning that the promoter is specifically expressed in the nervous system. Examples of such promoters include, without limitation, those isolated from the genes from myelin basic protein (MBP), glial fibrillary acid protein (GFAP), synapsins (e.g. human sysnapsin 1 gene promoter), neuron specific enolase (NSE), HB9 promoter and the promoter of CYP46A1 gene.

In one embodiment, said promoter is the promoter of the CYP46A1 gene.

In one embodiment, said promoter is cell type-specific, meaning that the promoter is only expressed in certain cell-types. Said promoter may be used for inducing the expression of the molecule of interest in specific cell-types, such as neurons or glia.

In one embodiment, the AAV vector according to the present invention further comprises inverted terminal repeats (ITR) sequences from any AAV serotype flanking the nucleic acid sequence encoding the molecule of interest. In one embodiment, said ITR sequence is from AAV2. In one embodiment, said ITR sequence is from AAV9.

As used herein, ITR sequences are regions found at each end of the AAV genome which function together in cis as origins of DNA replication and as packaging signals for the virus. ITRs, together with the AAV rep coding region, provide for the efficient excision and rescue from, and integration of a nucleotide sequence interposed between two flanking ITRs into a mammalian cell genome.

In one embodiment, the AAV vector according to the present invention comprises a nucleic acid sequence encoding a molecule of interest, a backbone of an AAV vector with ITR sequences derived from AAV2 and a CAG promoter.

An example of an AAV vector coding for ARSA is provided with sequence SEQ ID NO: 15.

In one embodiment, the AAV vector according to the present invention enables the expression of a molecule of interest, that is present in a healthy subject but absent in a subject affected with a disease or condition. Thus, in one embodiment, the AAV vector according to the present invention induces the expression of a molecule of interest at a level similar to that of a healthy subject.

In one embodiment, the AAV vector according to the present invention enables to decrease the expression of a gene of interest, that is not expressed in a healthy subject but expressed in a subject affected with a disease or condition. Thus, in one embodiment, the AAV vector according to the present invention enables the expression of a molecule of interest that decreases the expression of a gene of interest at a level similar to that of a healthy subject.

In one embodiment, said molecule of interest is a molecule selected from the group comprising or consisting of proteins (e.g. enzymes, antibodies, antibody fragments), peptides and nucleic acids (e.g. RNA interference molecules, such as siRNA or miRNA, antisense oligonucleotides).

In one embodiment, said molecule of interest is a protein, preferably an enzyme.

In one embodiment, said molecule of interest is arylsulfatase A (ARSA). In one embodiment, said molecule of interest is human ARSA. In one embodiment, said molecule of interest comprises or consists of the sequence SEQ ID NO: 9, or any protein having an amino acid sequence sharing at least 60, 65, 70, 75, 80, 85, 90, 95, 96, 97, 98, 99% or more identity with SEQ ID NO: 9.

SEQ ID NO: 9
MSMGAPRSLLLALAAGLAVARPPNIVLIFADDLGYGDLGCYGHPSSTT
PNLDQLAAGGLRFTDFYVPVSLCTPSRAALLTGRLPVRMGMYPGVLVP
SSRGGLPLEEVTVAEVLAARGYLTGMAGKWHLGVGPEGAFLPPHQGFH
RFLGIPYSHDQGPCQNLTCFPPATPCDGGCDQGLVPIPLLANLSVEAQ
PPWLPGLEARYMAFAHDLMADAQRQDRPFFLYYASHHTHYPQFSGQSF
AERSGRGPFGDSLMELDAAVGTLMTAIGDLGLLEETLVIFTADNGPET
MRMSRGGCSGLLRCGKGTTYEGGVREPALAFWPGHIAPGVTHELASSL
DLLPTLAALAGAPLPNVTLDGFDLSPLLLGTGKSPRQSLFFYPSYPDE
VRGVFAVRTGKYKAHFFTQGSAHSDTTADPACHASSSLTAHEPPLLYD
LSKDPGENYNLLGGVAGATPEVLQALKQLQLLKAQLDAAVTFGPSQVA
RGEDPALQICCHPGCTPRPACCHCPDPHA

Said ARSA is encoded by the ARSA gene. An example of a nucleic acid encoding ARSA, in particular human ARSA, is SEQ ID NO: 10.

Thus, in one embodiment, the AAV vector according to the present invention comprises a nucleic acid sequence comprising or consisting of SEQ ID NO: 10 or any nucleic acid sequence sharing at least 60, 65, 70, 75, 80, 85, 90, 95, 96, 97, 98, 99% or more identity with SEQ ID NO: 10.

In one embodiment, said molecule of interest is cholesterol 24-hydroxylase. In one embodiment, said molecule of interest is human cholesterol 24-hydroxylase. In one embodiment, said molecule of interest comprises or consists of the sequence SEQ ID NO: 11, or any protein having an amino acid sequence sharing at least 60, 65, 70, 75, 80, 85, 90, 95, 96, 97, 98, 99% or more identity with SEQ ID NO: 11.

SEQ ID NO: 11
MSPGLLLLGSAVLLAFGLCCTFVHRARSRYEHIPGPPRPSFLLGHLPC
FWKKDEVGGRVLQDVFLDWAKKYGPVVRVNVFHKTSVIVTSPESVKKF
LMSTKYNKDSKMYRALQTVFGERLFGQGLVSECNYERWHKQRRVIDLA
FSRSSLVSLMETFNEKAEQLVEILEAKADGQTPVSMQDMLTYTAMDIL
AKAAFGMETSMLLGAQKPLSQAVKLMLEGITASRNTLAKFLPGKRKQL
REVRESIRFLRQVGRDWVQRRREALKRGEEVPADILTQILKAEEGAQD
DEGLLDNFVTFFIAGHETSANHLAFTVMELSRQPEIVARLQAEVDEVI
GSKRYLDFEDLGRLQYLSQVLKESLRLYPPAWGTFRLLEEETLIDGVR
VPGNTPLLFSTYVMGRMDTYFEDPLTFNPDRFGPGAPKPRFTYFPFSL
GHRSCIGQQFAQMEVKVVMAKLLQRLEFRLVPGQRFGLQEQATLKPLD
PVLCTLRPRGWQPAPPPPPC

Cholesterol 24-hydroxylase is encoded by the CYP64A1 gene. A cDNA sequence for CYP46A1 is disclosed in Genbank Access Number AF094480 (SEQ ID NO: 12).

Thus, in embodiment, the AAV vector according to the present invention comprises a nucleic acid sequence comprising or consisting of SEQ ID NO: 12 or any nucleic acid sequence sharing at least 60, 65, 70, 75, 80, 85, 90, 95, 96, 97, 98, 99% or more identity with SEQ ID NO: 12.

In one embodiment, said molecule of interest is N-acetyl-alpha-glucosaminidase (NAGLU). In one embodiment, said molecule of interest is human NAGLU. In one embodiment, said molecule of interest comprises or consists of the sequence SEQ ID NO: 16, or any protein having an amino acid sequence sharing at least 60, 65, 70, 75, 80, 85, 90, 95, 96, 97, 98, 99% or more identity with SEQ ID NO: 16.

SEQ ID NO: 16
MEAVAVAAAVGVLLLAGAGGAAGDEAREAAAVRALVARLLGPGPAADF
SVSVERALAAKPGLDTYSLGGGGAARVRVRGSTGVAAAAGLHRYLRDF
CGCHVAWSGSQLRLPRPLPAVPGELTEATPNRYRYYQNVCTQSYSFVW
WDWARWEREIDWMALNGINLALAWSGQEAIWQRVYLALGLTQAEINEF
FTGPAFLAWGRMGNLHTWDGPLPPSWHIKQLYLQHRVLDQMRSFGMTP
VLPAFAGHVPEAVTRVFPQVNVTKMGSWGHFNCSYSCSFLLAPEDPIF
PIIGSLFLRELIKEFGTDHIYGADTFNEMQPPSSEPSYLAAATTAVYE
AMTAVDTEAVWLLQGWLFQHQPQFWGPAQIRAVLGAVPRGRLLVLDLF
AESQPVYTRTASFQGQPFIWCMLHNFGGNHGLFGALEAVNGGPEAARL
FPNSTMVGTGMAPEGISQNEVVYSLMAELGWRKDPVPDLAAWVTSFAA
RRYGVSHPDAGAAWRLLLRSVYNCSGEACRGHNRSPLVRRPSLQMNTS
IWYNRSDVFEAWRLLLTSAPSLATSPAFRYDLLDLTRQAVQELVSLYY
EEARSAYLSKELASLLRAGGVLAYELLPALDEVLASDSRFLLGSWLEQ
ARAAAVSEAEADFYEQNSRYQLTLWGPEGNILDYANKQLAGLVANYYT
PRWRLFLEALVDSVAQGIPFQQHQFDKNVFQLEQAFVLSKQRYPSQPR
GDTVDLAKKIFLKYYPRWVAGSW

Said NAGLU is encoded by the NAGLU gene. In one embodiment, the AAV vector according to the present invention comprises a nucleic acid sequence encoding for NAGLU, preferably human NAGLU.

An example of a nucleic acid encoding for human NAGLU is provided with SEQ ID NO: 17. Thus, in embodiment, the AAV vector according to the present invention comprises a nucleic acid sequence comprising or consisting of SEQ ID NO: 17 or any nucleic acid sequence sharing at least 60, 65, 70, 75, 80, 85, 90, 95, 96, 97, 98, 99% or more identity with SEQ ID NO: 17.

The present invention further relates to a composition comprising, consisting essentially of or consisting of an AAV vector as defined hereinabove.

As used herein, “consisting essentially of”, with reference to a composition, means that the AAV vector is the only one therapeutic agent or agent with a biologic activity within said composition.

The present invention further relates to a pharmaceutical composition comprising, consisting essentially of or consisting of an AAV vector as defined hereinabove and at least one pharmaceutically acceptable excipient.

The term “pharmaceutically acceptable excipient” includes any and all solvents, dispersion media, coatings, antibacterial and antifungal agents, isotonic and absorption delaying agents and the like. Said excipient does not produce an adverse, allergic or other untoward reaction when administered to an animal, preferably a human. For human administration, preparations should meet sterility, pyrogenicity, and general safety and purity standards as required by regulatory offices, such as, for example, FDA Office or EMA.

Pharmaceutically acceptable excipients that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

In one embodiment, the pharmaceutical compositions according to the present invention comprise vehicles which are pharmaceutically acceptable for a formulation capable of being injected to a subject. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The present invention further relates to a medicament comprising, consisting essentially of or consisting of an AAV vector as defined hereinabove.

The present invention further relates to a method for treating a disease or a condition in a subject in need thereof, comprising administering the recombinant AAV vector, the composition, the pharmaceutical composition, or the medicament as defined hereinabove to the subject.

In one embodiment, said disease or condition affects the nervous system.

In one embodiment, said disease or condition is a neurodegenerative disease. In one embodiment, said disease or condition is a neurodegenerative disease selected from the group comprising or consisting of Alzheimer's disease, Huntington's disease, Parkinson's disease and spinocerebellar ataxia.

In one embodiment, said disease or condition is Alzheimer's or Huntington's disease.

In one embodiment, said disease or condition is a neurodegenerative disease as described hereinabove and the molecule of interest to be expressed is cholesterol 24-hydroxylase.

Thus, in one embodiment, the present invention relates to a method for treating a neurodegenerative disease selected from the group comprising or consisting of Alzheimer's disease, Huntington's disease, Parkinson's disease and spinocerebellar ataxia in a subject in need thereof, comprising administrating to said subject a recombinant AAV vector comprising a nucleic acid sequence encoding cholesterol 24-hydroxylase, or a composition, pharmaceutical composition or medicament comprising said AAV vector. In one embodiment, said recombinant AAV vector comprises an AAV capsid protein comprising the amino acid sequence TLAVPFK (SEQ ID NO: 1).

In one embodiment, said disease or condition is a cancer. In one embodiment, said disease or condition is a glioblastoma.

In one embodiment, said disease or condition is a glioblastoma and the molecule of interest to be expressed is cholesterol 24-hydroxylase.

Thus, in one embodiment, the present invention relates to a method for treating a glioblastoma in a subject in need thereof, comprising administrating to said subject a recombinant AAV vector comprising a nucleic acid sequence encoding cholesterol 24-hydroxylase, or a composition, pharmaceutical composition or medicament comprising said AAV vector. In one embodiment, said recombinant AAV vector comprises an AAV capsid protein comprising the amino acid sequence TLAVPFK (SEQ ID NO: 1).

In one embodiment, said disease or condition is Alzheimer's disease, Huntington's disease, Parkinson's disease, spinocerebellar ataxia or glioblastoma and the molecule of interest to be expressed is cholesterol 24-hydroxylase.

Thus, in one embodiment, the present invention relates to a method for treating Alzheimer's disease, Huntington's disease, Parkinson's disease, spinocerebellar ataxia or glioblastoma in a subject in need thereof, comprising administrating to said subject a recombinant AAV vector comprising a nucleic acid sequence encoding cholesterol 24-hydroxylase, or a composition, pharmaceutical composition or medicament comprising said AAV vector. In one embodiment, said recombinant AAV vector comprises an AAV capsid protein comprising the amino acid sequence TLAVPFK (SEQ ID NO: 1).

In one embodiment, said disease or condition is a lysosomal storage disease (LSD).

In one embodiment, said disease or condition is metachromatic leukodystrophy (MLD).

As used herein, MLD is a LSD caused by an inherited deficiency of arylsulfatase A. Three clinical forms of MLD have been described, based on the age of symptom onset: late infantile, juvenile and adult forms.

Thus, in one embodiment, said MLD is selected from the group consisting of the late infantile form, the juvenile form and the adult form.

In one embodiment, the MLD is the late infantile form. Clinical manifestation of late infantile MLD begins up to 30 months of age. This form of MLD is considered the most severe, characterized by lack of or minimal residual ARSA activity, which entails rapid neurodegeneration.

In one embodiment, the MLD is the juvenile form. The juvenile form develops between the ages of 3 and 16 and is characterized by a less pronounced clinical manifestation in comparison with the late infantile form

In one embodiment, the MLD is the adult form. Clinical manifestation of the adult form of MLD begins in late adolescence, usually after 16 years of age. Adult MLD is the less severe form of the disease.

In one embodiment, said MLD is a late-onset form, preferably wherein the first symptoms occurred after 4 years.

In one embodiment, said MLD is an early-onset form, preferably wherein the first symptoms occurred before 4 years.

In one embodiment, said MLD is a rapidly progressive form of the disease.

In one embodiment, said disease or condition is MLD and the molecule of interest to be expressed is ARSA.

Thus, in one embodiment, the present invention relates to a method for treating MLD in a subject in need thereof, comprising administrating to said subject a recombinant AAV vector comprising a nucleic acid sequence encoding ARSA, or a composition, pharmaceutical composition or medicament comprising said AAV vector. In one embodiment, said recombinant AAV vector comprises an AAV capsid protein comprising the amino acid sequence TLAVPFK (SEQ ID NO: 1).

In one embodiment, the recombinant AAV vector comprising a nucleic acid sequence encoding ARSA inhibits or prevents sulfatide storage in a subject affected with MLD. Thus, the present invention further relates to a method for inhibiting or preventing sulfatide storage in a subject affected with MLD, comprising administering to the subject a recombinant AAV vector comprising a nucleic acid sequence encoding ARSA, or a composition, pharmaceutical composition, or medicament comprising said AAV vector.

In one embodiment, the recombinant AAV vector comprising a nucleic acid sequence encoding ARSA inhibits or prevents neuroinflammation, such as, for example, astrogliosis and/or microgliosis, in a subject affected with MLD. Thus, the present invention further relates to a method for inhibiting or preventing neuroinflammation, such as, for example, astrogliosis and/or microgliosis, in a subject affected with MLD, comprising administering to the subject a recombinant AAV vector comprising a nucleic acid sequence encoding ARSA, or a composition, pharmaceutical composition, or medicament comprising said AAV vector.

In one embodiment, said disease or condition is a mucopolysaccharidosis (MPS), preferably mucopolysaccharidosis type III (MPS III).

As used herein, MPS III is characterized by heparitin sulfate in the urine, progressive mental retardation, mild dwarfism, and other skeletal disorders. There are four clinically indistinguishable but biochemically distinct forms, each due to a deficiency of a different enzyme: type A caused by heparan N-sulfatase deficiency, type B caused by Alpha-N-acetylglucosaminidase deficiency, type C caused by acetyl-CoA:alpha-glucosaminide N-acetyltransferase deficiency and type D caused by N-acetylglucosamine-6-sulfatase deficiency.

In one embodiment, said disease or condition is a MPS III selected from the group consisting of type A, type B, type C and type D. In one embodiment, said disease or condition is MPS III type A. In one embodiment, said disease or condition is MPS III type B. In one embodiment, said disease or condition is MPS III type C. In one embodiment, said disease or condition is MPS III type D.

The present invention further relates to a method for increasing or inducing expression of a molecule of interest in the nervous system of a subject, comprising administering to said subject the recombinant AAV vector, the composition, the pharmaceutical composition, or the medicament as defined hereinabove. In one embodiment, said recombinant AAV vector comprises an AAV capsid protein comprising the amino acid sequence TLAVPFK (SEQ ID NO: 1). In one embodiment, said molecule of interest is ARSA. In one embodiment, said molecule of interest is cholesterol 24-hydroxylase. In one embodiment, said molecule of interest is NAGLU.

For uses in the methods described hereinabove, the AAV vector, the composition, the pharmaceutical composition, or the medicament according to the present invention is formulated for administration to the subject. Examples of routes of administrations, preferably routes of administration that require the crossing of the blood-brain barrier, are provided hereinbelow.

In one embodiment, said subject is a mammal. In one embodiment, said subject is a primate. In one embodiment, said subject is a human.

In one embodiment, said subject is a male. In one embodiment, said subject is a female. In one embodiment, said subject is an adult, i.e. equal or above the age of 18. In one embodiment, said subject is a child, i.e. below the age of 18. In one embodiment, said subject is a child below 18, 17, 16, 15, 14, 13, 12, 11, 10, 9, 8, 7, 6, 5, 4, 3, 2, 1 year(s) of age. In one embodiment, said subject is a child below 2 years of age. In one embodiment, said subject is healthy.

In one embodiment, said subject is affected or diagnosed with a disease or condition of the nervous system.

In one embodiment, said subject is affected or diagnosed with a neurodegenerative disease, preferably a neurodegenerative disease selected from the group comprising or consisting of Alzheimer's disease, Huntington's disease, Parkinson's disease and spinocerebellar ataxia.

In one embodiment, said subject is affected or diagnosed with a cancer. In one embodiment, said subject is affected or diagnosed with a glioblastoma. I

In one embodiment, said subject is affected or diagnosed with a lysosomal storage disease, preferably MLD.

In one embodiment, said subject is affected or diagnosed with the late infantile form, the juvenile form or the adult form of MLD. In one embodiment, said subject is affected or diagnosed with the late infantile form of MLD. In one embodiment, said subject is affected or diagnosed with the juvenile form of MLD. In one embodiment, said subject is affected or diagnosed with the adult form of MLD.

In one embodiment, said subject affected or diagnosed with MLD is pre-symptomatic, meaning that said subject does not present symptoms of the disease.

In one embodiment, said subject affected or diagnosed with MLD is symptomatic. In one embodiment, said subject affected or diagnosed with MLD is early symptomatic. As used herein, said subject is early symptomatic if said subject presents with signs of clinical manifestations but does not present diffuse areas of demyelination.

In one embodiment, said subject presents at least one of the symptoms described hereinbelow.

Examples of symptoms of MLD include, without limitation, sulfatide storage, myelin abnormalities on MRI, neuroinflammation, motor impairment and coordination loss.

In one embodiment, said subject is affected or diagnosed with mucopolysaccharidosis (MPS), preferably mucopolysaccharidosis type III (MPS III). In one embodiment, said subject is affected or diagnosed with a MPS III selected from the group consisting of type A, type B, type C and type D.

In one embodiment, said subject affected or diagnosed with MPS III is symptomatic.

In one embodiment, said subject affected or diagnosed with MPS III is presymptomatic.

For uses in the methods described hereinabove, the AAV vector, the composition, the pharmaceutical composition, or the medicament according to the present invention is formulated for administration to the subject.

In one embodiment, the AAV vector, the composition, the pharmaceutical composition, or the medicament according to the present invention is to be administered by any route of administration that requires the crossing of the blood-brain barrier. In one embodiment, the AAV vector, the composition, the pharmaceutical composition, or the medicament according to the present invention is not to be administered by a route that does not require the crossing of the blood-brain barrier.

Examples of routes of administration that require the crossing of the blood-brain include, without being limited to, buccal, nasal, oral, rectal, intramuscular, intravenous or subcutaneous administrations, or a combination thereof.

In one embodiment, the AAV vector, the composition, the pharmaceutical composition, or the medicament according to the present invention is to be administered buccally, nasally, orally, rectally, intramuscularly, intravenously or subcutaneously to a subject, or a combination thereof. In one embodiment, the AAV vector, the composition, the pharmaceutical composition, or the medicament according to the present invention is to be administered intravenously to the subject.

Examples of routes of administrations that do not require the crossing of the blood-brain barrier include, without being limited to, intrathecal or intracerebral administrations.

In one embodiment, the AAV vector, the composition, the pharmaceutical composition, or the medicament according to the present invention is to be administered with a combination of a route of administrations that does not require the crossing of the blood-brain barrier and a route of administrations that requires the crossing of the blood-brain barrier. An example of such combination is the administration by both intracerebral and intravenous administrations. Intrathecal or intra cerebroventricular deliveries could also be envisaged in combination with intravenous delivery.

Examples of forms adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In one embodiment, the AAV vector, the composition, the pharmaceutical composition, or the medicament according to the present invention is to be administered in a therapeutically effective amount.

The specific therapeutically effective dose level for any particular patient will depend upon a variety of factors including the disease being treated and the severity of the disease; activity of the AAV vector, the composition, the pharmaceutical composition, or the medicament according to the present invention employed; the age, body weight, general health, sex and diet of the subject; the time and route of administration of the AAV vector, the composition, the pharmaceutical composition, or the medicament according to the present invention employed; drugs used in combination or coincidental with the AAV vector, the composition, the pharmaceutical composition, or the medicament according to the present invention employed; and like factors well known in the medical arts.

For example, it is well within the skill of the art to start doses of the compound at levels lower than those required to achieve the desired therapeutic effect and to gradually increase the dosage until the desired effect is achieved. The total dose required for each treatment may be administered by multiple doses or in a single dose.

In one embodiment, the dose of AAV vectors that is to be administered to the subject is comprised between about 1×10¹⁰ and about 1×10¹⁶ vector genome, preferably between about 1×10¹¹ and about 1×10¹⁵ vector genome, more preferably between about 1×10¹² and about 1×10¹⁴ vector genome, even more preferably between 1×10¹³ and 1×10¹⁴ vector genome.

In one embodiment, the recombinant AAV vector according to the present invention is the only one therapeutic agent to treat or prevent a disease or condition as described hereinabove. Thus, in one embodiment, the recombinant AAV vector according to the present invention is to be used as a monotherapy.

In one embodiment, the recombinant AAV vector according to the present invention is to be administered in combination with another therapeutic agent.

In one embodiment, the other therapeutic agent is an immunosuppressive agent. As used herein, immunosuppressive agents are drugs that inhibit or prevent activity of the immune system.

In one embodiment, the other therapeutic agent is an anti-inflammatory agent. As used herein, anti-inflammatory agents are drugs that reduces inflammation (redness, swelling, and pain) in the body.

In one embodiment, the other therapeutic agent is selected from the group comprising or consisting of corticosteroid treatment, tacrolimus, sirolimus, cyclophosphamide, rituximab, fludarabine, or a combination thereof.

In one embodiment, the other therapeutic agent may be administered before, after or concomitantly with the administration of the recombinant AAV vector, the composition, the pharmaceutical composition, or the medicament according to the present invention.

For uses in the methods of the present invention, the crossing of the blood-brain barrier of the AAV vector may be improved by adding ultrasounds to the subject.

Thus, the present invention further relates to a method as described hereinabove, wherein said method further comprises exposing the subject with ultrasounds, preferably low frequency ultrasounds.

In one embodiment, the method according to the present invention comprises i) a step of administering the recombinant AAV vector, the composition, the pharmaceutical composition, or the medicament as defined hereinabove buccally, nasally, orally, rectally, intramuscularly, intravenously or subcutaneously to the subject or a combination thereof, preferably intravenously, and ii) a step of exposing the subject with ultrasounds, preferably low frequency ultrasounds.

Said exposure with ultrasounds may be done before, after or concomitantly with the administration of the recombinant AAV vector, the composition, the pharmaceutical composition, or the medicament according to the present invention.

In one embodiment, said ultrasounds have a frequency of about 100 to about 500 hHz, preferably of about 200 to about 400 hHz, more preferably to about 300 hHz.

### EXAMPLES

The present invention is further illustrated by the following examples.

### Example 1: Complete Correction of Brain and Spinal Cord Pathology in MLD Mice

### Materials and Methods

### Adeno-Associated Viral Vector Construction and Production

AAV vectors were produced and purified by Atlantic Gene therapies (Translational Vector Core Research grade services, Nantes, France). AAVPHP.eB-CAG ARSA-HA was produced by cloning the HA tag to the ARSA sequence under the CAG promoter. The viral constructs for pAAVPHP.eB-CAG-hARSA-HA contained the expression cassette consisting of the human ARSA genes, driven by a CMV early enhancer/chicken b-actin (CAG) synthetic promoter surrounded by inverted terminal repeats (ITR) sequences of AAV2. Plasmid for AAVPHP.eB was obtained from Addgene (United States). The final titer of the batch was 4·10¹² vector genomes (vg)/ml.

### Animal Model

All animal studies were performed in accordance with local and national regulations and were reviewed and approved by the relevant institutional animal care and use committee. ARSA-deficient mice (KO ARSA mice) were bred from homozygous founders on a 129/Ola strain and heterozygous mice were generated to be control mice. Mice were housed in a pathogen free animal facility in a temperature-controlled room and maintained on a 12-h light/dark cycle. Food and water were available ad libitum.

### Injection of AAV Vector

Female and male KO ARSA mice were anesthetized by isoflurane (2% induction). Animals were injected at 6 months of age or 9 months of age by intravenous retro-orbital delivery with saline (NaCl 0.9%) solution or AAVPHP.eB-hARSA-HA (5·10¹¹ vg total). Three groups of animals were performed: wildtype (WT, n=3), untreated (NT, n=8) and treated (AAV, n=8 at 6 months and n=8 at 9 months) KO ARSA mice. For each group, males and females were equally divided so that treatment efficacy is evaluated in both genders. The injected dose was determined according to previous results of dose ranging study on WT animals and evaluation of transduction efficacy.

### Tissue Preparation

Animals were sacrificed by an intraperitoneal administration of a lethal dose of Euthasol (180 mg/kg, Vetcare) 3 months after treatment. Mice were perfused intracardiacally with phosphate buffered saline (PBS). Brain, spinal cord, sciatic nerve, heart, liver, gall gladder, lung, spleen and kidney were collected for analysis. Different structures of a cerebral hemisphere (cortex, striatum, cerebellum, pons and rest of brain) were dissected and frozen in liquid nitrogen. Sciatic nerve, heart, liver, lung, spleen and kidney were directly frozen in liquid nitrogen and stored in −80° C. For DNA and protein extraction from the same samples, tissue samples were crushed in liquid nitrogen and divided into two equals parts. A cerebral hemisphere, a portion of spinal cord, sciatic nerve and gall bladder were post-fixed overnight in 4% paraformaldehyde (PFA)/PBS1×. Samples were rinsed three times in PBS 1× and cryoprotected in 30% sucrose/PBS1×. Tissue are embedded Tissue-Tek OCT compound (VWR International) and cut into 14-mm sagittal section of brain or transversal section of spinal cord or 4 mm longitudinal section of sciatic nerve or transversal section of gall bladder in cryostat (Leica, Langham, Tex.). Cryosections were dried at room temperature and stored at −20° C.

### Quantitative PCR

DNA was extracted from brain, spinal cord and peripheral organs using chloroform/phenol protocol. AAVPHP.eB-hARSAHA vector genome copy numbers were measured by quantitative PCR in cortex, striatum, cerebellum, pons, rest of brain, spinal cord and peripheral organs using the Light Cycler 480 SYBR Green I Master (Roche, France). The results (vector genome copy number per cell) were expressed as n-fold differences in the transgene sequence copy number relative to the Adck3 gene copy as internal standard (number of viral genome copy for 2N genome). Primers sequence for qPCR were: human Arsa (forward 5′-TCA CTG CAG ACA ATG GAC CTG A-3′ (SEQ ID NO: 5), reverse 5′-ACC GCC CTC GTA GGT CGT T-3′ (SEQ ID NO: 6)) and Adck3 (forward 5′-CCA CCT CTC CTA TGG GCA GA-3′ (SEQ ID NO: 7), reverse 5′-CCG GGC CTT TTC AAT GTC T-3′ (SEQ ID NO: 8)).

### Protein Extraction and ARSA Expression and Activity Quantification

Samples were homogenized in 0.3 ml of lysis buffer (100 mM Trizma base, 150 mM NaCl, 0.3% Triton; pH 7) and incubated for 30 min on ice and centrifuged. The supernatant was collected for the determination of (1) protein content (bicinchoninic acid [BCA] protein assay kit; Pierce Biotechnology/Thermo Fisher Scientific, Rockville, Ill.); (2) ARSA activity, using the artificial p-nitrocatechol sulfate (pNCS) substrate assay (Sigma-Aldrich, France). Assays were performed in triplicate and results are expressed as nanomoles of 4-nitrocatechol (4NC) per hour per milligram of protein. And (3) the concentration of recombinant hARSA using an indirect sandwich ELISA specific for human ARSA, using 2 specific noncommercial antibodies (from Dr Ulrich Matzner, Bonn). Assays were performed in duplicate and results are expressed as nanograms of hARSA per milligram of protein. All samples were quantified in duplicates.

### Histopathology

To evaluate sulfatide storage, frozen sections were postfixed in 4% PFA, stained with Alcian blue (A5268; Sigma-Aldrich) (0.05% in 0.025 M sodium acetate buffer, pH 5.7, containing 0.3 M MgCl2 and 1% PFA), rinsed in the same buffer without dye, counterstained with fast red (229113; Sigma-Aldrich) and mounted.

Immunohistochemical labeling was performed with the ABC method. Briefly, tissue sections are treated with peroxide (0.9% H2O2/0.3% Triton/PBS) for 30 min to inhibit endogenous peroxidase. Following washes with PBS, sections are incubated with the blocking solution (10% goat serum in PBS/0.3% TritonX-100) for 1 hr. The primary antibodies [rabbit anti-Calbindin (CB38; Swant, 1:10 000); mouse anti-GFAP (G3893, Sigma-Aldrich; 1:400); rabbit anti-Iba1 (019-19741, Wako; 1:500)] are diluted in blocking solution and incubated on tissue sections overnight at 4° C. After washes in PBS, sections are sequentially incubated with goat anti-rabbit or anti-mouse antibody conjugated to biotin (Vector Laboratories) for 30 min at room temperature, followed by the ABC complex (Vector Laboratories). After washes in PBS, the peroxidase activity is detected with diaminobenzidine as chromogen (Dako, Carpinteria, Calif.). In some cases, slides are counterstained with hematoxylin. The slides are mounted with Eukitt (VWR International). Slices are acquired at 20× by using a slide scanner (NanoZoomer2.ORS, Hamamatsu).

Tissue cryosections were permeabilized with PBS/0.3% TritonX-100 for 15 min and saturated with PBS/0.3% Triton/10% horse serum (HS) for 45 min. The primary antibodies were diluted in the saturation solution and incubated 1 h at 37° C. After washes in PBS/0.1% Triton, the secondary antibodies and DAPI were diluted in PBS/0.1% Triton/10% HS and added for 1 h at room temperature. After washes in PBS/0.1% Triton, the slides were mounted with fluorescent mounting medium (F4680; Sigma-Aldrich). Primary antibodies for immunofluorescence were rabbit polyclonal anti-hARSA (from V. Gieselmann and U. Matzner, Bonn, Germany; 1:1,000) and mouse anti-Lamp1 (1D4B, DSHB, 1:200). Secondary antibodies were diluted 1:1,000 and were donkey anti-rabbit/AlexaFluor 594 and anti-mouse/AlexaFluor488. Pictures were taken with a Confocal SP8 Leica DLS Inverted (Leica). For all images, brightness and contrast were adjusted with Image J software after acquisition to match with the observation. All histological studies were assessed blinded by two investigators.

### Stereological Cell Counts

Stereological counts were performed by two independent investigators, blind for both genotypes and treatments, using Image J software. All quantifications were done on three sections of brain and of spinal cord for each animal (n=3 9. Annual Congress of the French Society of Cell and Gene Therapy), adapted for LC-MS.

Determination of sulfatide isoforms was performed by scanning m/z 97 precursor by infusing 1:9 dilutions in chloroform-methanol (2:1) of brain sample lipid extracts at 10 ll/min. This allowed the identification of 23 sulfatide species ranging from C16 to C26, with the following m/z mass of the [M-H]— ion in multiple re-action-monitoring (MRM) mode: C16:0 (m/z=778.6), C16:0-OH (m/z=794.6), C18:0 (m/z=806.6), C18:0-D3 (m/z=809.6), C18:1-OH (m/z=820.6), C18:0-OH (m/z=822.6), C20:0 (m/z=834.6), C20:1-OH (m/z=848.6), C20:0-OH (m/z=850.6), C22:1 (m/z=860.6), C22:0 (m/z=862.6), C23:0-OH or C22:1-OH (m/z=876.6), C22:0-OH (m/z=878.6), C24:1 (m/z=888.6), C24:0 (m/z=890.6), C23:0 (m/z=OH 892.6), C25:1 (m/z=902.6), C25:0-OH or C24:1-OH (m/z=904.6), C24:0-OH (m/z=906.6), C26:1 (m/z=916.6), C26:0 (m/z=918.6), C25:0-OH (m/z=920.6), C26:1-OH (m/z=932.6) and C26:0-OH (m/z=934.6).

### Statistical Analysis

Data were analyzed using GraphPad Prism 8 software. The statistical significance of values among groups was evaluated by ANOVA, followed by the least significant difference t-test. All values used in figures and text are expressed as mean standard error of the mean (SEM). Differences were considered significant at p<0.05. For all graphs, a special symbol has been assigned for each individual so that it is possible to correlate between ARSA expression and astrogliosis, sulfatide accumulation and so.

### Results

Validation of pAAV-CAG-hARSA-HA Plasmid In Vitro

After hARSA-HA cloning in the pAAV plasmid and validation by sequencing, an in vitro assay based on 293T cells transfection with pAAV-CAG-hARSA-HA plasmid was performed. We demonstrated hARSA-HA expression using HA staining, in transfected cells as well as a significant increase in ARSA activity in supernatant of transfected cells, up to 90-folds compared to non-transfected cells, 72 h after transfection. These data confirmed the functionality of the pAAV-CAG-hARSA-HA plasmid. The AAVPHP.eB-hARSA-HA vector was produced as described.

Widespread Distribution and Expression of AAVPHP.eB-hARSA-HA in the CNS of KO ARSA Mice

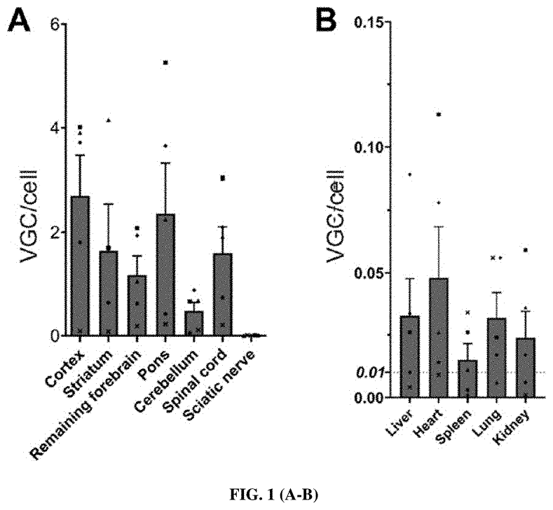

Treated KO ARSA mice (n=8) mice received a single intravenous injection of AAVPHP.eB-hARSA-HA. Treatment was well tolerated, no adverse event was observed in mice injected with the AAVPHP.eB vector, attesting for the safety of the procedure. Vector injection resulted in a widespread transduction of CNS, with a mean of 2.71±0.76 vector genome copies per cell (VGC) in the cortex, 1.6 VGC±0.90 in the striatum, 2.3 VGC±0.96 in the pons, 1.2 VGC±0.37 in the remaining forebrain, 0.5 VGC±0.17 in the cerebellum and 1.6 VGC±0.50 in the spinal cord (FIG. 1A) in treated mice. The mean number of VGC in peripheral organs was less than 0.05 VGC, indicating a low peripheral transduction of the vector (FIG. 1B).

In accordance with the biodistribution profile, hARSA expression was detected by immunofluorescence studies in several areas of brain of treated KO ARSA mice, such as striatum, hippocampus, thalamus, corpus callosum, pons and cerebellum. Moreover, hARSA-positive cells were also detected in the spinal cord of treated mice. As a negative control, hARSA protein expression was not detected in untreated KO ARSA mice. To be active, ARSA enzyme needs to be targeted to the lysosome. Proper lysosomal localization was confirmed by co-staining with anti-hARSA and anti-Lamp1 (lysosomal marker) antibodies, performed on brain and spinal cord sections of treated KO ARSA mice. A colocalization of hARSA and Lamp1 was observed in different areas of CNS, indicating that hARSA is correctly localized in lysosomes and thus could catabolize sulfatides.

hARSA Activity and Expression in CNS of Treated KO-ARSA Mice

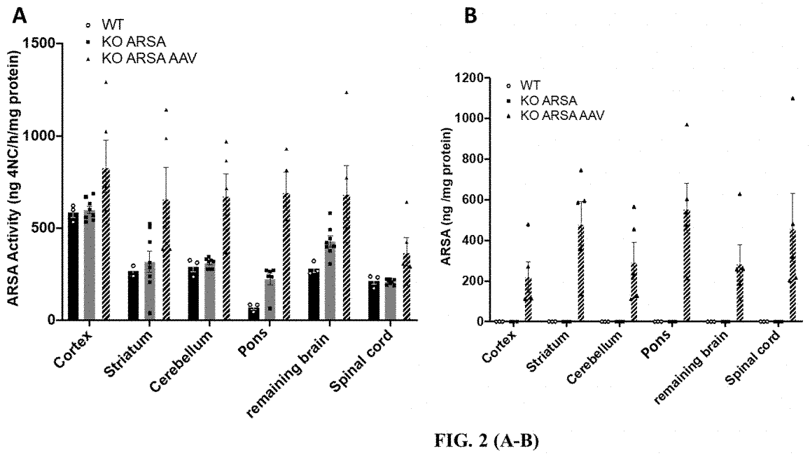

To validate the functionality of recombinant hARSA in treated KO ARSA mice, ARSA activity was measured in different structures of the CNS. We demonstrated a clear trend to ARSA over activity in the cortex, pons, cerebellum and spinal cord in treated KO ARSA mice (FIG. 2A). Moreover, expression of recombinant hARSA was assessed by ELISA in several structures of brain and the spinal cord with a mean to 326 ng ARSA/mg protein in treated KO mice whereas it was not detected in WT and untreated mice (FIG. 2B). We demonstrated a high hARSA expression in the brain and the spinal cord in treated KO ARSA. To conclude, treated mice with AAVPHP.eB-hARSA-HA express high levels of functional ARSA enzyme.

### Complete Correction of Sulfatide Storage in CNS of Treated KO ARSA

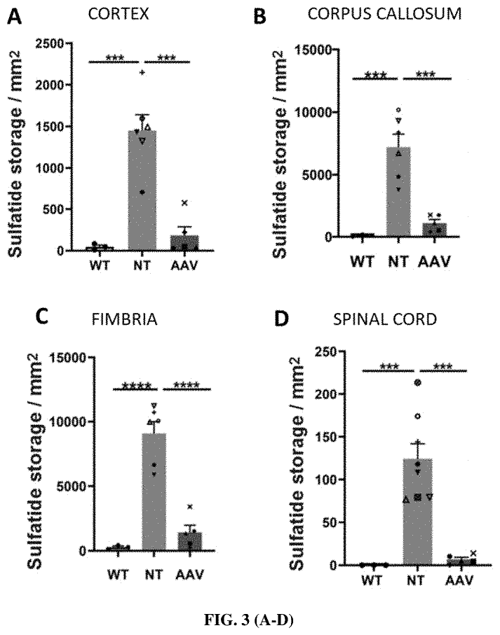

Sulfatide accumulation starts during fetal development, is obviously detectable at 3 months of age in the CNS of KO ARSA mice (corpus callosum and pons) and then increases progressively with age. To assess the efficiency of intravenous administration of AAVPHP.eBARSA-HA vector to decrease sulfatide storage, alcian staining was performed in the brain, spinal cord, sciatic nerve and gall bladder sections of untreated and treated KO ARSA animals and compared to wild-type control. Nine-month-old untreated KO ARSA mice display massive sulfatide storage in brain, spinal cord, sciatic nerve and gall bladder compared to WT mice. In 9-month-old treated animals, 3 months after intravenous injection, AAVPHP.eBARSA-HA vector had significantly decreased the sulfatide storage in brain and spinal cord of treated KO ARSA mice. This was confirmed by the quantification of the number of sulfatide storage inclusions in the cortex, corpus callosum, fimbria and spinal cord (FIGS. 3A-D). Indeed, a tremendous decrease of sulfatide accumulation was observed in treated mice that were almost similar to WT mice for brain and spinal cord. A complete correction of sulfatides accumulation was also observed in the cerebellum of KO ARSA mice treated with a lower dose of the AAVPHP.eB-ARSA-HA vector, i.e. 2.5·10¹¹ vg total, administered intravenously at 6 months and analyzed at 12 months.

AAVPHP.eB-hARSA-HA Treatment Rescue Neuroinflammation in MLD Mouse Model

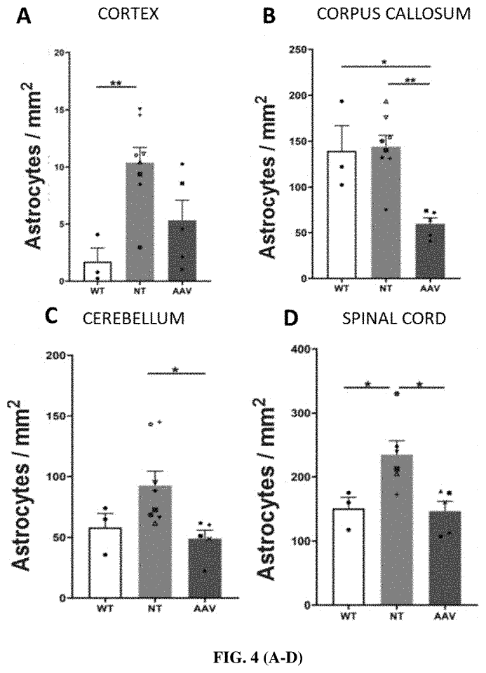

Astrogliosis and microgliosis are two hallmarks of MLD pathology that are present in MLD mouse model. To assess the effect of AAVPHP.eB-hARSA-HA treatment on astrogliosis and microgliosis in KO ARSA mice, an immunohistochemical staining was performed with anti-GFAP or anti-Iba1 antibodies on the brain and spinal cord sections of different groups of animals. A significant increase of GFAP-positive cells was observed in cortex and spinal cord of 9-month-old untreated mice compared to WT mice (FIGS. 4A, 4D). In the cerebellum, an increase of GFAP-positive cells was also detected in KO ARSA mice, compared to WT animals, even if not significant (FIG. 4C). No astrogliosis was detected in the corpus callosum of untreated mice (FIG. 4B). Three months after injection with AAVPHP.eB-hARSA-HA, a significant decrease of astrogliosis was observed in the spinal cord of treated KO ARSA mice, as well as a trend to improvement in the cortex and cerebellum, vouching for a clear therapeutic effect (FIGS. 4A-D). A significant increase of Iba1-positive cells was observed in cortex and corpus callosum of untreated KO ARSA mice, compared to WT mice (FIGS. 5A-B). Three months after AAVPHP.eB-hARSA-HA injection, a significant decrease in microgliosis was observed in both cortex and corpus callosum of treated mice (FIGS. 5A-B), indicating a positive therapeutic effect. In summary, markers of neuroinflammation observed in the CNS of 9-month-old KO ARSA mice were significantly reduced after intravenous administration of the AAVPHP.eB-hARSA-HA therapeutic vector.

Characterization of 9-month-old KO ARSA mice and Complete Correction of Sulfatide Storage in CNS

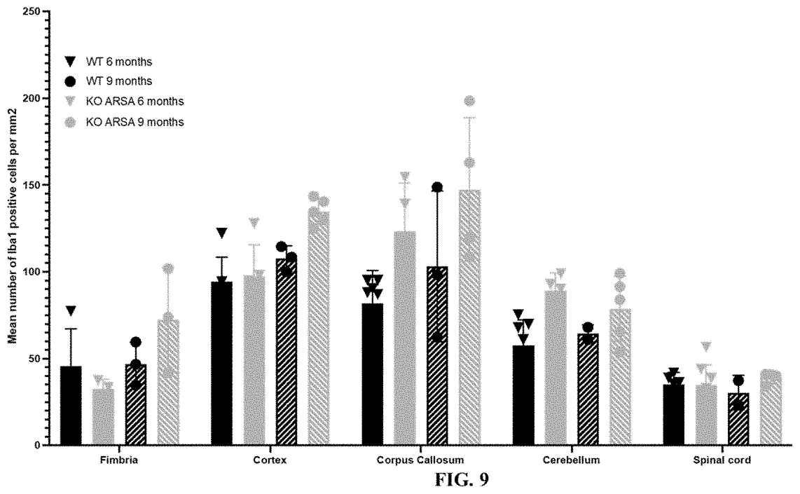

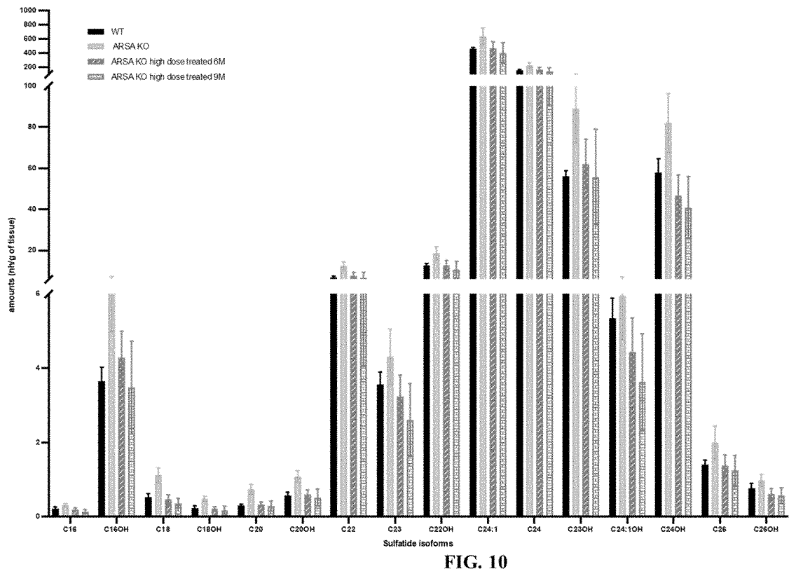

Then, the aim was to evaluate the efficacy of the intravenous administration of the AAVPHP.eB-hARSA-HA therapeutic vector in older animals, i.e. 9-month-old KO ARSA mice, that present more severe alterations than 6-month KO ARSA mice. To do so, the sulfatides storage and the microgliosis were first characterized in 9-month-old KO ARSA mice to evaluate the sulfatide storage and the neuroinflammation at injection time. As shown in FIG. 8, there was an increased sulfatides storage in 9-month-old KO ARSA mice as compared to 6-month-old KO ARSA mice in several nervous system areas, including, for example, fimbria, cortex, corpus callosum or spinal cord. By contrast, the sulfatide storage was very similar in the peripheral organs in 9-month-old KO ARSA mice and 6-month-old KO ARSA mice. Regarding microgliosis, there was an increased microgliosis in 9-month-old KO ARSA mice as compared to 6-month-old KO ARSA mice in several nervous system areas, including, for example, fimbria or cortex (FIG. 9). Altogether, these data confirm that the 9-month-old KO ARSA mice present more severe alterations than the 6-month-old KO ARSA mice.

Then, the efficacy of the intravenous administration of the AAVPHP.eB-hARSA-HA therapeutic vector in reducing sulfatides storage was assessed in 9-month-old KO ARSA mice for several sulfatide isoforms. As shown in FIG. 10, ARSA KO mice presented an increased sulfatides storage for all sulfatide isoforms tested. A significant decrease of all species of sulfatide was observed in the cerebellum of treated mice both injected at 6 and 9 months that were almost similar to WT mice (FIG. 10). A significant decrease was also observed in the spinal cord of treated mice both injected at 6 and 9 months. Thus, these data show that the intravenous administration of the AAVPHP.eB-hARSA-HA therapeutic vector can restore the sulfatides levels of animals with a more severe form of the disease.

Blue alcian stainings also demonstrated an efficient correction of the sulfatide storage in all treated group in cerebellum and in spinal cord of all treated animals.

### Example 2: Intravenous Injection of AAV-PHP.eB-ARSA-HA in Monkeys

### Materials and Methods

### Adeno-Associated Viral Vector Construction and Production

AAV vectors were produced and purified by Atlantic Gene therapies (Translational Vector Core Research grade services, Nantes, France). AAVPHP.eB-CAG ARSA-HA was produced by cloning the HA tag to the ARSA sequence under the CAG promoter. The viral constructs for pAAVPHP.eB-CAG-hARSA-HA contained the expression cassette consisting of the human ARSA genes, driven by a CMV early enhancer/chicken b-actin (CAG) synthetic promoter surrounded by inverted terminal repeats (ITR) sequences of AAV2. Plasmid for AAVPHP.eB was obtained from Addgene (United States). The final titer of the batch was 1·10¹³ vector genomes (vg)/ml.

### Animal Model

All animal studies were performed in accordance with local and national regulations and were reviewed and approved by the relevant institutional animal care and use committee. NHP were housed in a temperature- and humidity-controlled animal facility (target temperature 20-24° C., no specific humidity target in the guidelines (20 to 60%) with a 12-h light-dark cycle (no record for light). NHP diet pellets (ref 307, Safe) were given ad libitum except during the fasting experimental period and each day animal received one fruit or one vegetable and seeds or dry fruits. Tap water will be offered ad libitum in polycarbonate bottles.

### Injection of AAV Vector

Two females monkey received 1×10¹³ vg total dose of AAVPHP.eB-CAG-ARSA-HA in the saphenous vein. Then, the monkey was treated with corticoids for 8 days (from D-1 to D7) following intravenous injection. The monkeys were followed-up during 6 weeks, with blood sampling at 3 and 6 weeks. ARSA concentration was measured in the cerebrospinal fluid and in various tissues. In addition a female monkey received 1×10¹³ vg total dose of AAVPHP.eB-CAG-ARSA-HA intrathecally and was followed for 3 weeks post injection.

A control animal that received AAVPHP.eB at the same concentration but with another transgene not related to ARSA was used as a negative control for ARSA expression to evaluate background expression.

### Tissue Preparation

Animals were sacrificed by an intravenous administration of a lethal dose of Euthasol (140 mg/kg, Vetcare) 6 weeks after treatment. NHP was perfused intracardiacally with phosphate buffered saline (PBS), followed by 500 mL of PFA 4%. Brain, spinal cord, sciatic nerve, heart, liver, gall gladder, lung, spleen and kidney were collected for analysis. Different structures of a cerebral hemisphere (frontal, temporal and occipital cortex, caudate, putamen, hippocampus, thalamus, corpus callosum, cerebellar white matter, pons) were dissected and frozen in liquid nitrogen. Spinal cord, sciatic nerve, dorsal root ganglia (DRG) heart, liver, lung, spleen and kidney were directly frozen in liquid nitrogen and stored in −80° C. For protein extraction from the same samples, tissue samples were crushed in liquid nitrogen and divided into two equals parts.

The brain was divided in 2 and one hemisphere was devoted to histological analysis and included in paraffin as well as a portion of spinal cord, sciatic nerve were post-fixed overnight in 4% paraformaldehyde (PFA)/PBS1×. Samples were rinsed three times in PBS 1× and embedded in paraffin and cut into 5-um sagittal section of brain or transversal section of spinal cord or 5 um lcoronal section for the brain.

### Protein Extraction and ARSA Expression and Activity Quantification

Samples were homogenized in 0.3 ml of lysis buffer (100 mM Trizma base, 150 mM NaCl, 0.3% Triton; pH 7) and incubated for 30 min on ice and centrifuged. The supernatant was collected for the determination of (1) protein content (bicinchoninic acid [BCA] protein assay kit; Pierce Biotechnology/Thermo Fisher Scientific, Rockville, Ill.); (2) ARSA activity, using the artificial p-nitrocatechol sulfate (pNCS) substrate assay (Sigma-Aldrich, France). Assays were performed in triplicate and results are expressed as nanomoles of 4-nitrocatechol (4NC) per hour per milligram of protein. And (3) the concentration of recombinant hARSA using an indirect sandwich ELISA specific for human ARSA, using 2 specific noncommercial antibodies (from Pr. Gielselmann, Bonn). Assays were performed in duplicate and results are expressed as nanograms of hARSA per milligram of protein. All samples were quantified in duplicates.

### Results

The tolerance and efficacy of the injection of the AAVPHP.eB-CAG-ARSA-HA vector was assessed in non-human primates (NHP).

The tested NHP, i.e. two with an intravenous injection of the vector and one with an intrathecal injection of the vectors, showed a good tolerance of the procedure. Indeed, the weight of the animals was not affected throughout the duration of the experiments (Table 1 below). In addition, extensive evaluation of cervical, thoracic and lumbar DRG structure of the tested NHPs with intravenous delivery showed no signs of toxicity.

TABLE 1

| | Monkey 1 (IV) | Monkey 3 (IV) | Monkey 4 (IT) |
|---|---|---|---|
| Baseline 1 | 2.75 | 3.15 | 3.4 |
| Baseline 2 | 2.85 | 3.25 | 3.5 |
| Surgery | 2.85 | 3.2 | 3.8 |
| 1 week post | — | 3.15 | 3.7 |
| operation | | | |
| 3 weeks post | 2.9 | 3.25 | 3.5 |
| operation | | | |
| 6 weeks post | 2.9 | 3.25 | — |
| operation | | | |

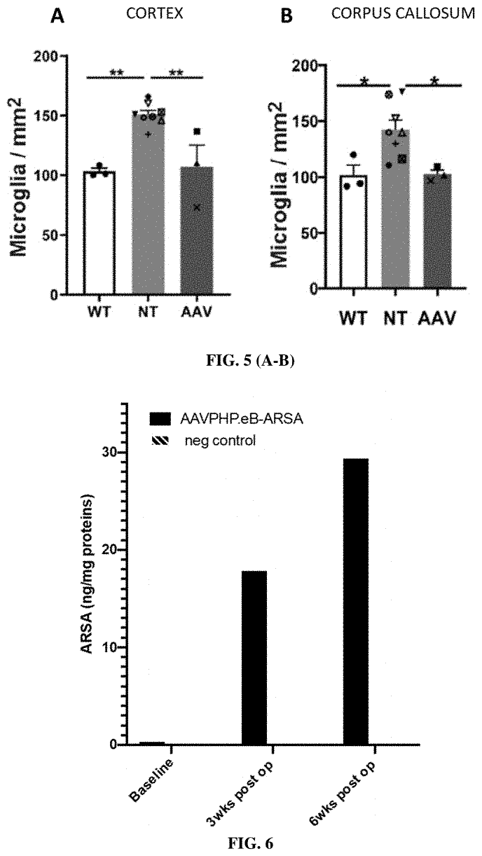

Regarding efficacy, as shown in FIG. 6, exogenous ARSA was detected in the cerebrospinal fluid of the monkey having received an injection of AAVPHP.eB-CAG-ARSA-HA in the saphenous vein but not in the control monkey.

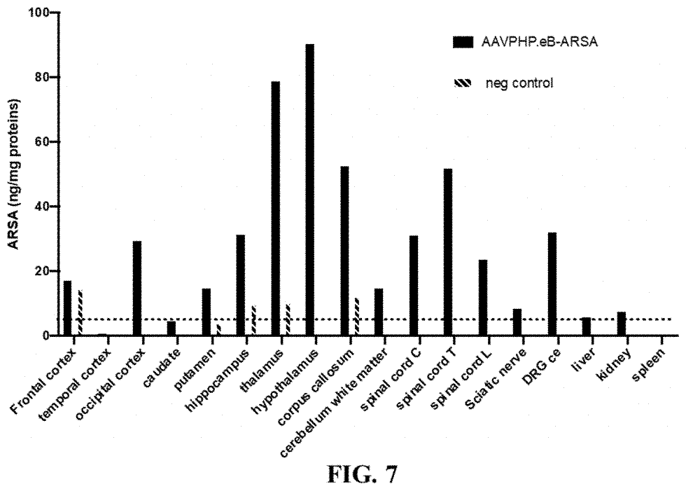

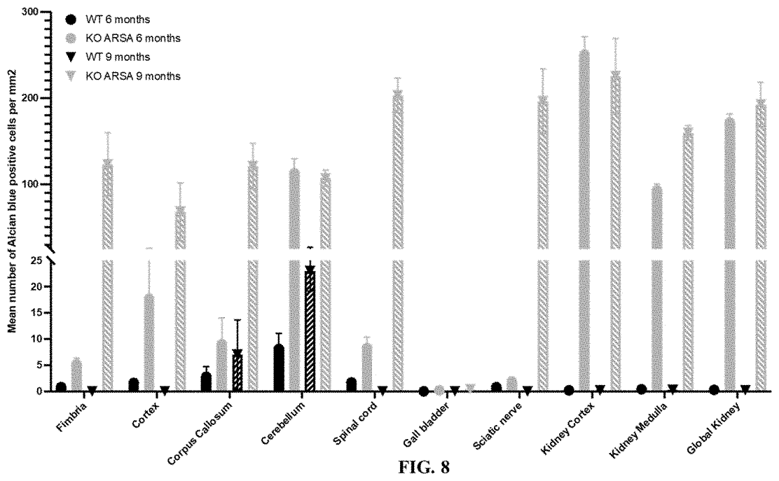

In addition, the injection of AAVPHP.eB-CAG-ARSA-HA in the saphenous vein of the monkey resulted in a widespread transduction of the nervous system, including expression in the corpus callosum, the cortex, the thalamus, the hypothalamus, the hippocampus and the spinal cord (FIG. 7). By contrast, ARSA was poorly detected in the peripheral organs.

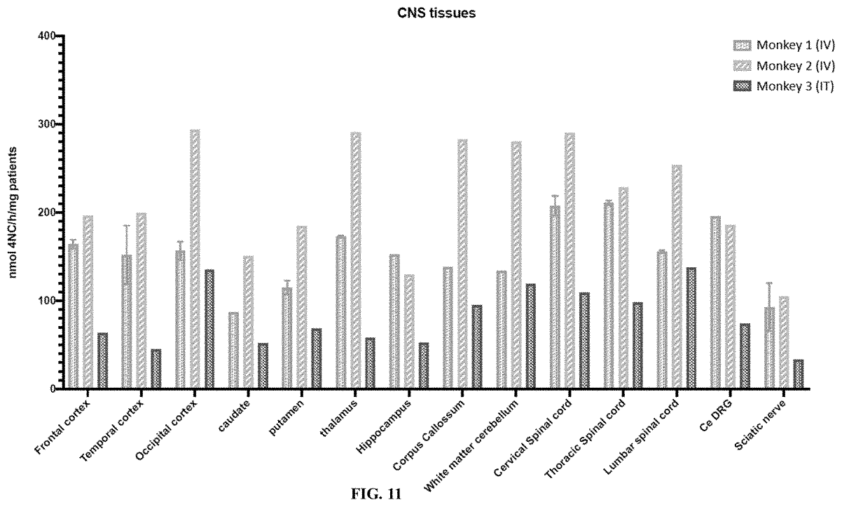

The ARSA activity was next compared between the NHPs having received an intravenous injection of AAVPHP.eB-CAG-ARSA-HA and the NHP having received an intrathecal injection of AAVPHP.eB-CAG-ARSA-HA. As shown in FIG. 11, the intravenous injection of AAVPHP.eB-CAG-ARSA-HA in NHP induced a wide expression of ARSA in the nervous system, and in particular, enabled the expression of ARSA in nervous system areas that were poorly targeted by the intrathecal administration of the vector, such as, for example, the cortex, the spinal cord and the dorsal root ganglia. Thus, the intravenous administration of the vector enables to express ARSA in large areas of the nervous system.

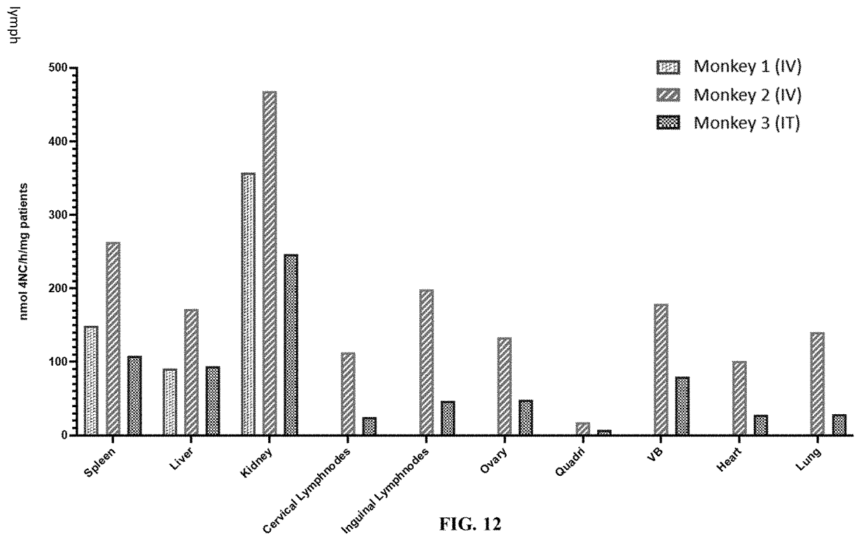

Interestingly, this large expression of ARSA in the nervous system was not associated with a massive expression of ARSA in peripheral tissues (FIG. 12), showing thereof that the intravenous injection of the vector enables to target the nervous system with a limited impact in the peripheral organs.

Altogether, these data demonstrate that the intravenous injection of AAV-PHP.eB inducing the expression of a protein of interest, such as ARSA, results in a wide expression of the protein in the nervous system of NHP, thereby confirming the capability of the vector to cross the blood-brain barrier and its transduction efficiency in the central nervous system.

1. A method for treating a disease or condition affecting the nervous system in a subject in need thereof, comprising administrating to said subject a recombinant AAV vector comprising a nucleic acid sequence encoding a molecule of interest,
wherein said recombinant AAV vector comprises an AAV capsid protein comprising the amino acid sequence TLAVPFK (SEQ ID NO: 1),
wherein said recombinant AAV vector is administered buccally, nasally, orally, rectally, intramuscularly, intravenously or subcutaneously or a combination thereof to said subject, and
wherein said disease or condition and said molecule of interest are one of the followings combinations:
i) the disease or condition is metachromatic leukodystrophy (MLD), and the molecule of interest is arylsulfatase A (ARSA);
ii) the disease or condition is Alzheimer's disease, Huntington's disease, Parkinson's disease, spinocerebellar ataxia or glioblastoma, and the molecule of interest is cholesterol 24-hydroxylase;
iii) the disease or condition is mucopolysaccharidosis type III, and the molecule of interest is N-acetyl-alpha-glucosaminidase.

2. The method according to claim 1, wherein said subject is a primate.

3. The method according to claim 2, wherein said subject is a human.

4. The method according to claim 1, wherein said AAV vector is a variant AAV9 vector.

5. The method according to claim 4, wherein the amino acid sequence TLAVPFK (SEQ ID NO: 1) is inserted between the amino acid residues 588-589 of the AAV9 capsid protein of sequence SEQ ID NO: 2.

6. The method according to claim 5, wherein said AAV9 capsid protein further comprises at least one of the mutations A587D and Q588G.

7. The method according to claim 6, wherein said AAV9 capsid protein further comprises the two mutations A587D and Q588G.

8. The method according to claim 1, wherein the disease or condition is metachromatic leukodystrophy (MLD) and the molecule of interest is ARSA.

9. The method according to claim 8, wherein the MLD is selected from the group consisting of the late infantile form, the juvenile form, and the adult form.

10. The method according to claim 1, wherein said subject is symptomatic.

11. The method according to claim 10, wherein said subject presents at least one of the following symptoms: sulfatide storage, myelin abnormalities on MRI, neuroinflammation, motor impairment and/or coordination loss.

12. The method according to claim 1, wherein said method further comprises a step of exposing the subject to ultrasounds.

13. A method for increasing or inducing expression of a molecule of interest in the nervous system of a subject, comprising administrating to said subject a recombinant AAV vector comprising a nucleic acid sequence encoding the molecule of interest,
wherein said recombinant AAV vector comprises an AAV capsid protein comprising the amino acid sequence TLAVPFK (SEQ ID NO: 1),
wherein said recombinant AAV vector is administered buccally, nasally, orally, rectally, intramuscularly, intravenously or subcutaneously to said subject,
wherein said molecule of interest is ARSA.

14. The method according to claim 13, wherein said subject is a primate.

15. The method according to claim 14, wherein said subject is a human.

16. The method according to claim 13, wherein said AAV vector is a variant AAV9 vector.

17. The method according to claim 16, wherein the amino acid sequence TLAVPFK (SEQ ID NO: 1) is inserted between the amino acid residues 588-589 of the AAV9 capsid protein of sequence SEQ ID NO: 2.

18. The method according to claim 17, wherein said AAV9 capsid protein further comprises the two mutations A587D and Q588G.

19. The method according to claim 13, wherein said method further comprises a step of exposing the subject to ultrasounds.


SEQUENCE LISTING (17 sequences)

SEQ ID NO: 1 - protein, 7 aa, Artificial Sequence
  heterologous peptide
TLAVPFK

SEQ ID NO: 2 - protein, 736 aa, Artificial Sequence
  AAV9 VP1 capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 743 aa, Artificial Sequence
  AAV-PHP.B capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 4 - protein, 743 aa, Artificial Sequence
  AAV-PHP.eB capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - DNA, 22 bp, Artificial Sequence
  Primer ARSA
tcactgcagacaatggacctga

SEQ ID NO: 6 - DNA, 19 bp, Artificial Sequence
  Primer ARSA
accgccctcgtaggtcgtt

SEQ ID NO: 7 - DNA, 20 bp, Artificial Sequence
  Primer Adck3
ccacctctcctatgggcaga

SEQ ID NO: 8 - DNA, 19 bp, Artificial Sequence
  Primer Adck3
ccgggccttttcaatgtct

SEQ ID NO: 9 - protein, 509 aa, Homo sapiens
MSMGAPRSLLLALAAGLAVARPPNIVLIFADDLGYGDLGCYGHPSSTTPNLDQLAAGGLRFTDFYVPVSLCTPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 10 - DNA, 1600 bp, Homo sapiens
atgggggcaccgcggtccctcctcctggccctggctgctggcctggccgttgcccgtccgcccaacatcgtgctgatctttgccgacgacctcggctatggggacctgggctgctatgggcaccccagctctaccactcccaacctggaccagctggcggcgggagggctgcggttcacagacttctacgtgcctgtgtctctgtgcacaccctctagggccgccctcctgaccggccggctcccggttcggatgggcatgtaccctggcgtcctggtgcccagctcccgggggggcctgcccctggaggaggtgaccgtggccgaagtcctggctgcccgaggctacctcacaggaatggccggcaagtggcaccttggggtggggcctgagggggccttcctgcccccccatcagggcttccatcgatttctaggcatcccgtactcccacgaccagggcccctgccagaacctgacctgcttcccgccggccactccttgcgacggtggctgtgaccagggcctggtccccatcccactgttggccaacctgtccgtggaggcgcagcccccctggctgcccggactagaggcccgctacatggctttcgcccatgacctcatggccgacgcccagcgccaggatcgccccttcttcctgtactatgcctctcaccacacccactaccctcagttcagtgggcagagctttgcagagcgttcaggccgcgggccatttggggactccctgatggagctggatgcagctgtggggaccctgatgacagccataggggacctggggctgcttgaagagacgctggtcatcttcactgcagacaatggacctgagaccatgcgtatgtcccgaggcggctgctccggtctcttgcggtgtggaaagggaacgacctacgagggcggtgtccgagagcctgccttggccttctggccaggtcatatcgctcccggcgtgacccacgagctggccagctccctggacctgctgcctaccctggcagccctggctggggccccactgcccaatgtcaccttggatggctttgacctcagccccctgctgctgggcacaggcaagagccctcggcagtctctcttcttctacccgtcctacccagacgaggtccgtggggtttttgctgtgcggactggaaagtacaaggctcacttcttcacccagggctctgcccacagtgataccactgcagaccctgcctgccacgcctccagctctctgactgctcatgagcccccgctgctctatgacctgtccaaggaccctggtgagaactacaacctgctggggggtgtggccggggccaccccagaggtgctgcaagccctgaaacagcttcagctgctcaaggcccagttagacgcagctgtgaccttcggccccagccaggtggcccggggcgaggaccccgccctgcagatctgctgtcatcctggctgcaccccccgcccagcttgctgccattgcccagatccccatgccgaattcactcctcaggtgcaggctgcctatcagaaggtggtggctggtgtggccaatgccctggctcacaaataccact

SEQ ID NO: 11 - protein, 500 aa, Homo sapiens
MSPGLLLLGSAVLLAFGLCCTFVHRARSRYEHIPGPPRPSFLLGHLPCFWKKDEVGGRVLQDVFLDWAKKYGPVVRVNVFHKTSVIVTSPESVKKFLMSTKYNKDSKMYRALQTVFGERLFGQGLVSECNYERWHKQRRVIDLAFSRSSLVSLMETFNEKAEQLVEILEAKADGQTPVSMQDMLTYTAMDILAKAAFGMETSMLLGAQKPLSQAVKLMLEGITASRNTLAKFLPGKRKQLREVRESIRFLRQVGRDWVQRRREALKRGEEVPADILTQILKAEEGAQDDEGLLDNFVTFFIAGHETSANHLAFTVMELSRQPEIVARLQAEVDEVIGSKRYLDFEDLGRLQYLSQVLKESLRLYPPAWGTFRLLEEETLIDGVRVPGNTPLLFSTYVMGRMDTYFEDPLTFNPDRFGPGAPKPRFTYFPFSLGHRSCIGQQFAQMEVKVVMAKLLQRLEFRLVPGQRFGLQEQATLKPLDPVLCTLRPRGWQPAPPPPPC

SEQ ID NO: 12 - DNA, 1503 bp, Homo sapiens
atgagccccgggctgctgctgctcggtagcgccgtcctgctcgccttcggcctctgctgcaccttcgtgcaccgcgctcgcagccgctacgagcacatccccgggccgccgcggcccagtttccttctaggacacctcccctgcttttggaaaaaggatgaggttggtggccgtgtgctccaagatgtgtttttggattgggctaagaagtatggacctgttgtgcgggtcaacgtcttccacaaaacctcagtcatcgtcacgagtcctgagtcggttaagaagttcctgatgtcaaccaagtacaacaaggactccaagatgtaccgtgcgctccagactgtgtttggtgagagactcttcggccaaggcttggtgtccgaatgcaactatgagcgctggcacaagcagcggagagtcatagacctggccttcagccggagctccttggttagcttaatggaaacattcaacgagaaggctgagcagctggtggagattctagaagccaaggcagatgggcagaccccagtgtccatgcaggacatgctgacctacaccgccatggacatcctggccaaggcagcttttgggatggagaccagtatgctgctgggtgcccagaagcctctgtcccaggcagtgaaacttatgttggagggaatcactgcgtcccgcaacactctggcaaagttcctgccagggaagaggaagcagctccgggaggtccgggagagcattcgcttcctgcgccaggtgggcagggactgggtccagcgccgccgggaagccctgaagaggggcgaggaggttcctgccgacatcctcacacagattctgaaagctgaagagggagcccaggacgacgagggtctgctggacaacttcgtcaccttcttcattgctggtcacgagacctctgccaaccacttggcgttcacagtgatggagctgtctcgccagccagagatcgtggcaaggctgcaggccgaggtggatgaggtcattggttctaagaggtacctggatttcgaggacctggggagactgcagtacctgtcccaggtcctcaaagagtcgctgaggctgtacccaccagcatggggcacctttcgcctgctggaagaggagaccttgattgatggggtcagagtccccggcaacaccccgctcttgttcagcacctatgtcatggggcggatggacacatactttgaggacccgctgactttcaaccccgatcgcttcggccctggagcacccaagccacggttcacctacttccccttctccctgggccaccgctcctgcatcgggcagcagtttgctcagatggaggtgaaggtggtcatggcaaagctgctgcagaggctggagttccggctggtgcccgggcagcgcttcgggctgcaggagcaggccacactcaagccactggaccccgtgctgtgcaccctgcggccccgcggctggcagcccgcacccccaccacccccctgctga

SEQ ID NO: 13 - DNA, 8543 bp, Artificial Sequence
  AAV9-PHP. B capsid protein
ccaatgatacgcgtcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacatcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtggacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaatatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgggccctgctcgatgccctggccatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaaatgcatgaattcgatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaatcaagctatcaagtgccacctgacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagagatctgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctagcttcgatcaactacgcagacaggtaccaaaacaagtgttctcgtcacgtgggcattaatctgattctgtttccctgcagacaatgcgagagaatgaatcagaactcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaagccaggtatggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgcccaaactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagtcgacttgcttgttaatcaataaaccgtttaattcgtttcagttgaactttggtctctgcgaagggcaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatcacatgtcctaggaactatcgatccatcacactggcggccgctcgactagagcggccgccaccgcggtggagctccagcttttgcggaccgaatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 14 - DNA, 8543 bp, Artificial Sequence
  AAV9-PHP. eB capsid protein
ccaatgatacgcgtcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacatcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtggacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaatatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgggccctgctcgatgccctggccatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaaatgcatgaattcgatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaatcaagctatcaagtgccacctgacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagagatctgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctagcttcgatcaactacgcagacaggtaccaaaacaagtgttctcgtcacgtgggcattaatctgattctgtttccctgcagacaatgcgagagaatgaatcagaactcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaagccaggtatggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagtcgacttgcttgttaatcaataaaccgtttaattcgtttcagttgaactttggtctctgcgaagggcaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatcacatgtcctaggaactatcgatccatcacactggcggccgctcgactagagcggccgccaccgcggtggagctccagcttttgcggaccgaatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 15 - DNA, 6817 bp, Artificial Sequence
  Vector
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctctagaactagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatgtccatgggggcaccgcggtccctcctcctggccctggctgctggcctggccgttgcccgtccgcccaacatcgtgctgatctttgccgacgacctcggctatggggacctgggctgctatgggcaccccagctctaccactcccaacctggaccagctggcggcgggagggctgcggttcacagacttctacgtgcctgtgtctctgtgcacaccctctagggccgccctcctgaccggccggctcccggttcggatgggcatgtaccctggcgtcctggtgcccagctcccgggggggcctgcccctggaggaggtgaccgtggccgaagtcctggctgcccgaggctacctcacaggaatggccggcaagtggcaccttggggtggggcctgagggggccttcctgcccccccatcagggcttccatcgatttctaggcatcccgtactcccacgaccagggcccctgccagaacctgacctgcttcccgccggccactccttgcgacggtggctgtgaccagggcctggtccccatcccactgttggccaacctgtccgtggaggcgcagcccccctggctgcccggactagaggcccgctacatggctttcgcccatgacctcatggccgacgcccagcgccaggatcgccccttcttcctgtactatgcctctcaccacacccactaccctcagttcagtgggcagagctttgcagagcgttcaggccgcgggccatttggggactccctgatggagctggatgcagctgtggggaccctgatgacagccataggggacctggggctgcttgaagagacgctggtcatcttcactgcagacaatggacctgagaccatgcgtatgtcccgaggcggctgctccggtctcttgcggtgtggaaagggaacgacctacgagggcggtgtccgagagcctgccttggccttctggccaggtcatatcgctcccggcgtgacccacgagctggccagctccctggacctgctgcctaccctggcagccctggctggggccccactgcccaatgtcaccttggatggctttgacctcagccccctgctgctgggcacaggcaagagccctcggcagtctctcttcttctacccgtcctacccagacgaggtccgtggggtttttgctgtgcggactggaaagtacaaggctcacttcttcacccagggctctgcccacagtgataccactgcagaccctgcctgccacgcctccagctctctgactgctcatgagcccccgctgctctatgacctgtccaaggaccctggtgagaactacaacctgctggggggtgtggccggggccaccccagaggtgctgcaagccctgaaacagcttcagctgctcaaggcccagttagacgcagctgtgaccttcggccccagccaggtggcccggggcgaggaccccgccctgcagatctgctgtcatcctggctgcaccccccgcccagcttgctgccattgcccagatccccatgccgaattcactcctcaggtgcaggctgcctatcagaaggtggtggctggtgtggccaatgccctggctcacaaataccactgagcctacccttacgacgtccctgactacgcgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttatcgataccgtcgacctcgagggggggcccggtacccagcttttgttccctttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgggccgctgaagcttgcatgcctgcaggtcgactctagaggatccccgccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgcggcccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 16 - protein, 743 aa, Homo sapiens
  NAGLU
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSW

SEQ ID NO: 17 - DNA, 2232 bp, Homo sapiens
  Human NAGLU
atggaggcggtggcggtggccgcggcggtgggggtccttctcctggccggggccgggggcgcggcaggcgacgaggcccgggaggcggcggccgtgcgggcgctcgtggcccggctgctggggccaggccccgcggccgacttctccgtgtcggtggagcgcgctctggctgccaagccgggcttggacacctacagcctgggcggcggcggcgcggcgcgcgtgcgggtgcgcggctccacgggcgtggcggccgccgcggggctgcaccgctacctgcgcgacttctgtggctgccacgtggcctggtccggctctcagctgcgcctgccgcggccactgccagccgtgccgggggagctgaccgaggccacgcccaacaggtaccgctattaccagaatgtgtgcacgcaaagctactctttcgtgtggtgggactgggcccgctgggagcgagagatagactggatggcgctgaatggcatcaacctggcactggcctggagcggccaggaggccatctggcagcgggtgtacctggccttgggcctgacccaggcagagatcaatgagttctttactggtcctgccttcctggcctgggggcgaatgggcaacctgcacacctgggatggccccctgcccccctcctggcacatcaagcagctttacctgcagcaccgggtcctggaccagatgcgctccttcggcatgaccccagtgctgcctgcattcgcggggcatgttcccgaggctgtcaccagggtgttccctcaggtcaatgtcacgaagatgggcagttggggccactttaactgttcctactcctgctccttccttctggctccggaagaccccatattccccatcatcgggagcctcttcctgcgagagctgatcaaagagtttggcacagaccacatctatggggccgacactttcaatgagatgcagccaccttcctcagagccctcctaccttgccgcagccaccactgccgtctatgaggccatgactgcagtggatactgaggctgtgtggctgctccaaggctggctcttccagcaccagccgcagttctgggggcccgcccagatcagggctgtgctgggagctgtgccccgtggccgcctcctggttctggacctgtttgctgagagccagcctgtgtatacccgcactgcctccttccagggccagcccttcatctggtgcatgctgcacaactttgggggaaaccatggtctttttggagccctagaggctgtgaacggaggcccagaagctgcccgcctcttccccaactccaccatggtaggcacgggcatggcccccgagggcatcagccagaacgaagtggtctattccctcatggctgagctgggctggcgaaaggacccagtgccagatttggcagcctgggtgaccagctttgccgcccggcggtatggggtctcccacccggacgcaggggcagcgtggaggctactgctccggagtgtgtacaactgctccggggaggcctgcaggggccacaatcgtagcccgctggtcaggcggccgtccctacagatgaataccagcatctggtacaaccgatctgatgtgtttgaggcctggcggctgctgctcacatctgctccctccctggccaccagccccgccttccgctacgacctgctggacctcactcggcaggcagtgcaggagctggtcagcttgtactatgaggaggcaagaagcgcctacctgagcaaggagctggcctccctgttgagggctggaggcgtcctggcctatgagctgctgccggcactggacgaggtgctggctagtgacagccgcttcttgctgggcagctggctagagcaggcccgagcagcggcagtcagtgaggccgaggccgatttctacgagcagaacagccgctaccagctgaccttgtgggggccagaaggcaacatcctggactatgccaacaagcagctggcggggttggtggccaactactacacccctcgctggcggcttttcctggaggcgctggttgacagtgtggcccagggcatccctttccaacagcaccagtttgacaaaaatgtcttccaactggagcaggccttcgttctcagcaagcagaggtaccccagccagccgcgaggagacactgtggacctggccaagaagatcttcctcaaatattacccccgctgggtggccggctcttggtga